SEQUENCE LISTING (17 sequences)

SEQ ID NO: 1 - protein, 4 aa, Artificial Sequence
  myristoylation sequence
CXXX

SEQ ID NO: 2 - protein, 8 aa, Artificial Sequence
  epitope tag
DYKDDDDK

SEQ ID NO: 3 - protein, 25 aa, Artificial Sequence
  hydrophobic C-terminal sequence
GAGFATPVTLALVPALLATFWSLLL

SEQ ID NO: 4 - protein, 116 aa, Unknown
  Lynx1 mammalian prototoxin
MTHLLTVFLVALMPVAQALECHVCAYNDGDNCFKPMRCPAMATYCMTTRTYFTPYRMKVRKSCVPSCFETVYDGYSKHASATSCCQYYLCNGAGFATPVTLALVPALLATFWSLLL

SEQ ID NO: 5 - protein, 132 aa, Unknown
  alpha bungarotoxin
MSALLILALVGAAVADYKDDDDKLIVCHTTATSPISAVTCPPGENLCYRKMWCDAFCSSRGKVVELGCAATCPSKKPYEEVTCCSTDKCNPHPKQRPGAAAGGALCNGAGFATPVTLALVPALLATFWSLLL

SEQ ID NO: 6 - protein, 124 aa, Unknown
  kappa bungarotoxin
MSALLILALVGAAVADYKDDDDKLRTCLISPSSTPQTCPNGQDICFLKAQCDKFCSIRGPVIEQGCVATCPQFRSNYRSLLCCTTDNCNHAAAGGALCNGAGFATPVTLALVPALLATFWSLLL

SEQ ID NO: 7 - protein, 78 aa, unknown
  conotoxin PnIB
MSALLILALVGAAVAGCCSLPPCALSNPDYCAAADYKDDDDKIDAAAGGALCNGAGFATPVTLALVPALLATFWSLLL

SEQ ID NO: 8 - protein, 78 aa, Unknown
  conotoxin MII
MSALLILALVGAAVAGCCSNPVCHLEHSNLCAAADYKDDDDKIDAAAGGALCNGAGFATPVTLALVPALLATFWSLLL

SEQ ID NO: 9 - protein, 108 aa, Unknown
  conotoxin
MSALLILALVGAAVAACRKKWEYCIVPIIGFIYCCPGLICGPFVCVIDYKDDDDKLAAAGNGNGNGNGNGNGNGDGNGGALCNGAGFATPVTLALVPALLATFWSLLL

SEQ ID NO: 10 - protein, 102 aa, Unknown
  conotoxin
MSALLILALVGAAVACKGKGAKCSRLMYDCCTGSCRSGKCIDYKDDDDKLAAAGNGNGNGNGNGNGNGDGNGGALCNGAGFATPVTLALVPALLATFWSLLL

SEQ ID NO: 11 - protein, 32 aa, Torpedo shark
NQFLPKLLNATACDGELSSSGIIFYVLYLIFY

SEQ ID NO: 12 - protein, 42 aa, Unknown
  placenta alkaline phosphatase
TACDLAPPAGTTDAAHPGRSVVPALLPLLAGTLLLLETATAP

SEQ ID NO: 13 - protein, 41 aa, Unknown
  decay accelerating factor
HETTPNKGSGTTSGTTRLLSGHTCFLTTGLLGTLVTMGLLT

SEQ ID NO: 14 - protein, 35 aa, Trypanosoma brucei
EPEPEPEPEPEPGAATLKSVALPFAIAAAALVAAF

SEQ ID NO: 15 - protein, 36 aa, hamster
QKESQAYYDGRRSSAVLFSSPPVILLISFLIFLMVG

SEQ ID NO: 16 - protein, 44 aa, Rattus norvegicus
KTINVIRDKLVKCGGISLLVQNTSWLLLLLLSLSFLQATDFISI

SEQ ID NO: 17 - protein, 36 aa, Trypanosoma brucei
  variant surface glycoprotein
ESNCKWENNACKDSSILVTKKFALTVVSAAFVALLF

(19) United States
(12) Patent Application Publication
(10) Pub. No.: US 2006/0057614 A1
(43) Pub. Date: Mar. 16, 2006

(54) Tethering neuropeptides and toxins for modulation of ion channels and receptors

(76) Inventor: Nathaniel Heintz, Pelham Manor, NY (US)

Correspondence Address: KLAUBER & JACKSON, 411 HACKENSACK AVENUE, HACKENSACK, NJ, 07601, US

(21) Appl. No.: 11/197,766

(22) Filed: Aug. 4, 2005

Related U.S. Application Data
(60) Provisional application No. 60/598,664, filed on Aug. 4, 2004.

(51) Int. Cl. G01N 1/28; C12Q 1/68; G01N 33/53; C12M 1/34

(52) U.S. Cl. 435/6; 435/7.1; 435/287.2; 427/2.11

(57) ABSTRACT

The present invention provides a method for tethering peptides, including neuropeptides and peptide toxins, including naturally occurring and mutant or altered peptides, to a surface, including the cell surface. The tethered peptides are not naturally tethered or attached to a surface, including a cell surface, or through a cell membrane. The tethered neuropeptides and toxins are useful in the targeted modulation of synaptic transmission and for regulation of cellular physiology, including neurophysiology, in vitro, ex vivo and in vivo. The use of these tethered peptides, including neuropeptides and toxins, in therapeutic and diagnostic methods and screening assays is also provided.

### RELATED APPLICATIONS

The present Application claims the benefit of provisional Application U.S. Ser. No. 60/598,664, filed Aug. 4, 2004, under 35 U.S.C. § 119(e), the disclosure of which is hereby incorporated by reference in its entirety.

### GOVERNMENTAL SUPPORT

The research leading to the present invention was supported, at least in part, by a grant from the National Institutes of Health, Grant No NIH 1R21NS047751/01. Accordingly, the Government may have certain rights in the invention.

### FIELD OF THE INVENTION

The present invention relates generally to a method for tethering peptides, including neuropeptides and peptide toxins, including naturally occurring and mutant or altered peptides, to a surface, including the cell surface. The tethered neuropeptides and toxins are useful in the targeted modulation of synaptic transmission and for regulation of cellular physiology, including neurophysiology, in vitro, ex vivo and in vivo. The use of these tethered peptides, including neuropeptides and toxins, in therapeutic and diagnostic methods and screening assays is also provided.

### BACKGROUND OF THE INVENTION

The physiologies of cells, and their roles in complex multi-cellular organisms, depend on the electrical and chemical signals carried by a broad array of ion channels and receptors. Ion channels and receptors in the nervous system are naturally modulated by neurotransmitters, which include acetylcholine, biogenic amines (including dopamine, epinephrine, norepinephrine and serotonin), excitatory amino acids (including glutamate, glycine and g-aminobutiric acid (GABA) and neuropeptides (of which over 50 are known, the most numerous being amino acid neurotransmitters, including bradykinin, gastrin, secretin, oxytocin, gonadotropin-releasing hormone, beta-endorphin, enkephalin, substance P, somatostatin, prolactin, growth hormone-releasing hormone, bombesin, neurotensin, neuropeptide Y, vasopressin, angiotensin II, insulin, glucagon, thyrotropin-releasing hormone, vasoactive intestinal peptide). Neurotransmitters act on receptors and can act as inhibitory or excitatory signals, depending on the transmitter and particular receptor. The type and number of receptors and their subtypes is large and complex, even for any given neurotransmitter.

In addition and importantly, venomous animals, including elapid snakes, cone snails, spiders and bacteria, produce an enormous variety of peptide toxins that exert their toxic action by binding with high affinity to specific ion channels and receptors. The table below (TABLE 1) indicates a number of such neurotoxins, their source and actions.

TABLE 1

| NEUROTOXIN | SOURCE | NEURONAL ACTION |
|---|---|---|
| Agitoxin | Scorpion | Blocks potassium channels |
| alpha-bungarotoxin | Krait (snake) | Blocks acetylcholine (nicotinic) receptor |
| Anatoxin | Algae | Acetylcholine (Ach) receptor agonist |
| Apamine | Honey bee | Blocks potassium channels |
| Atracotoxin | Blue Mountain Funnel | Blocks voltage-gated calcium channels |
| | Web Spider | |
| Batrachotoxin | Poison Arrow Frog | Prevents sodium channels from closing |
| beta-bungarotoxin | Krait (snake) | Inhibits release of ACh at neuromuscular |
| | | junction and blocks potassium channels |
| Botulinum toxin | Bacteria | Blocks acetylcholine release |
| Brevetoxin | Red Tide Dinoflagellate | Activates sodium channels |
| Capsaicin | Cayenne Pepper | Excites peripheral nerve endings |
| Charybdotoxin | Scorpion | Blocks potassium channels |
| Ciguatoxin | Dinoflagellate | Opens sodium channels |
| Cobratoxin | Cobra | Blocks nicotinic receptors |
| Conotoxin | Marine Snail | Several types, blocking voltage-sensitive calcium |
| | | channels, voltage-sensitive sodium channels, and |
| | | ACh receptors |
| Crotoxin | S. American Rattlesnake | Reduces acetylcholine release |
| Dendrotoxin | Green Mamba | Blocks voltage-gated potassium channels |
| Domoic acid | Blue mussel | Glutamate/kainite receptor agonist |
| Erabutoxin | Sea snake | Blocks Ach (nicotinic) receptors |
| Grammotoxin SIA | South American Rose | Blocks calcium channels |
| | Tarantula | |
| Gonyautoxin | Dinoflagellate | Blocks sodium channels |
| Hainantoxin | Chinese bird spider | Blocks sodium channels |
| Holocyclotoxin | Australian paralysis tick | Inhibits release of acetylcholine |
| Homobatrachotoxin | Pitohui (bird) | Activates sodium channels |
| HWTX-1(huwentoxin-1) | Chinese bird spider | Blocks calcium channels |
| Iberiotoxin | Scorpion | Blocks potassium channels |
| Joro spider toxin | Joro spider | Blocks glutamate receptors |
| Kaliotoxin | Scorpion | Blocks potassium channels |
| Kurtoxin | South African Scorpion | Blocks calcium channels |
| Latrotoxin | Black Widow Spider | Enhances acetylcholine release |
| Maculotoxin | Blue-Ringed Octopus | Blocks sodium channels |
| Margatoxin | Scorpion | Blocks potassium channels |
| Noxiustoxin | Scorpion | Blocks sodium channels |
| Palytoxin | Soft coral | Activates sodium channels |
| Philanthotoxin | Predaceous Wasp | Blocks glutamate receptors |
| Phoneutriatoxin | Banana spider | Slows sodium channel inactivation |
| Phrixotoxin | Chilean fire tarantula | Blocks potassium channels |
| Robustotoxin | Funnel web spider | Opens sodium channels |
| Saxitoxin | Dinoflagellate | Blocks sodium channels |
| SNX-482 | African Tarantula | Blocks calcium channels |
| Stichodactyla Toxin | Sea Anemone | Blocks voltage-gated potassium channels |
| Taicatoxin | Australian Taipan snake | Inhibits voltage-gated calcium channels |
| Tetrodotoxin (TTX) | Pufferfish | Blocks sodium channels |
| Texilotoxin | Australian common | Blocks release of acetylcholine |
| | Brown snake | |
| Tityustoxin-K | Brazilian Scorpion | Blocks potassium channels |
| Versutoxin | Funnel web spider | Opens sodium channels |

Lynx1, is a new member of the Ly-6/α-bungarotoxin gene superfamily whose members contain a structural receptor binding motif characteristic of the neuroactive snake venom toxins, termed the three-fingered or toxin fold Miwa J M et al (1999) Neuron 23:105-114). Lynx1 is expressed in the deep nuclei of the cerebellum localized to discrete subfields of large projection neurons in several brain structures, including the soma and proximal dendrites of Purkinje neurons. Lynx 1, a mammalian prototoxin, has been shown to share with α-bungarotoxin the ability to bind and modulate nicotinic acetylcholine receptors (nAChRs) (Ibanez-Tallon, I et al (2002) Neuron 33:893-903). Unlike venom toxins, however, Lynx1 is a neuronal cell surface protein, specifically a membrane-anchored protein, that is tethered to the cell surface via a glycophospholipid (GPI) anchor. Lynx is described in PCT US99/21702, published as WO 00/17356.

Despite significant efforts into the study of ligand-gated channels and the apparent commercial success of certain drugs broadly targeting these receptors and their neurotransmitters, there remains a need in the art for a more specific understanding of the molecular mechanisms of action of these receptors and identification and manipulation of molecules involved in the mediation of the action of these receptors and their neurotransmitters. There remains a need in the art for more specific and selective receptor mediators both for the study of these receptors and for the advancement of therapeutic approaches aimed at these receptors and the treatment and amelioration of various disorders, including those of the CNS. It would be highly useful and applicable, in therapies and other methods, to harness the pharmacological properties of neurotransmitters, particularly neuropeptides and peptide toxins, to understand and manipulate their activities, including in therapeutic or prophylactic methods and for use in genetic experiments and neurological and pharmacological studies.

The citation of references herein shall not be construed as an admission that such is prior art to the present invention.

### SUMMARY OF THE INVENTION

In its broadest aspect, the present invention extends to a method for tethering toxins or peptides, particularly toxins or peptides which are not naturally tethered or attached to a cell surface or through a cell membrane, including naturally occurring and mutant or altered peptide sequences or non-peptide toxins, to a surface, including the cell surface. In a particular aspect, these toxins or peptides interact with, signal via, or otherwise modulate cell receptors or ion channels.

In accordance with the present invention, a method is provided for tethering neuropeptides and toxins, including naturally occurring and mutant or altered neuropeptides and toxins, to a surface, including the cell surface. In a particular aspect, these neuropeptides and toxins interact with, signal via, or otherwise modulate cell receptors or ion channels, particularly including neural cell receptors or ion channels, including for instance acetylcholine (nicotinic) receptors, glutamate receptors, serotonin receptors, GABA receptors, calcium channels, sodium channels and potassium channels.

In accordance with the present invention, a method is provided for tethering immune modulators, including naturally occurring and mutant or altered immune modulators, to a surface, including the cell surface. In a particular aspect, these immune modulators interact with, signal via, or otherwise modulate cell receptors or ion channels involved in the immune system or immune response, particularly including T cell receptors.

In accordance with the present invention, a method is provided for tethering cytokines, including naturally occurring and mutant or altered cytokines, to a surface, including the cell surface. In a particular aspect, these cytokines interact with, signal via, or otherwise modulate cell receptors or ion channels, particularly to generate an intracellular signal that modulates or alters cell growth or response, including in the hematopoietic system.

In accordance with the present invention, a method is provided for tethering hormones, including naturally occurring and mutant or altered hormones, to a surface, including the cell surface. In a particular aspect, these hormones interact with, signal via, or otherwise modulate cell receptors or ion channels, including for example leptin, which interacts with the leptin receptor DB.

In an aspect of the invention, the peptide, neuropeptide or toxin acts on a receptor or ion channel at the cell membrane and is expressed in tethered form in and on the cell expressing its receptor or ion channel. Thus, the tethered peptide acts in a cell autonomous fashion, modulating or signaling via its receptor or ion channel without the necessary addition of other or other types of cells.

In a further aspect of the invention, the peptide, neuropeptide or toxin acts on a receptor or ion channel at the cell membrane and is expressed in tethered form in and on a distinct cell, not expressing its receptor or ion channel. Thus, the tethered peptide acts in a cell dependent fashion, in trans, for instance acting on a synaptic partner, modulating or signaling via its receptor or ion channel upon the addition of other or other types of cells expressing its receptor or ion channel.

In one aspect, a neuroactive toxin, particularly selected from a spider, snake, scorpion, snail, bacteria, bee or fish toxin is tethered to the surface of a cell, particularly a neural cell or cell involved with or associated with the nervous system or nerve cell response.

The invention provides a tethered peptide attached to or integrated in a cell membrane, cell or other surface, which is not naturally tethered, membrane bound or membrane associated. The invention further provides a tethered neuropeptide or toxin which is attached to or integrated in a cell membrane, cell or other surface, which is not naturally tethered, membrane bound or membrane associated. The invention particularly provides a tethered conotoxin or bungarotoxin. In a further particular aspect, a tethered ω-o-conotoxin, α-bungarotoxin or κ-bungarotoxin is provided.

The neuropeptides and toxins may be tethered or otherwise attached to a cell membrane by various means, including utilizing known and natural sequences from membrane and/or cell surface proteins which are naturally attached to or transverse through the cell membrane, or any such other means known or available in the art to attach or associate a peptide or molecule with the membrane or cell surface, including via attachment to or association with a membrane protein. Sequences from known and natural membrane and/or cell surface proteins serve to transverse or attach to the membrane per se or are recognized and function to signal attachment and/or specific modification in cells. The peptides of the present invention may be tethered in accordance with the invention via a membrane attachment sequence. A membrane attachment sequence may be selected from a transmembrane domain, a hydrophobic domain, a PH domain, a GPI attachment sequence, a myristoylation sequence (Cys-A-A-X) (SEQ ID NO:1), a palmitoylation sequence, or any other such peptide sequence which encodes for or signals the attachment of a lipid or hydrophobic sequence or the association of a peptide with the membrane.

Thus, the invention includes a tethering cassette for expression of or preparation of a tethered peptide, comprising a promoter, a peptide to be tethered, a linker sequence and a membrane attachment sequence. In a particular embodiment, the cassette additionally comprises a secretion signal located in the cassette prior to the peptide to be tethered. In one embodiment, the secretion signal is a signal sequence. In one embodiment, the promoter is a cell specific promoter. In a further embodiment, the promoter is an inducible promoter. In a particular embodiment, the peptide to be tethered is selected from a neuropeptide, neurotoxin, immune modulator, hormone or cytokine.

The invention provides a tethering system for expression of or preparation of a tethered peptide, comprising a promoter, a peptide to be tethered, a linker sequence and a membrane attachment sequence, and further comprising a means for induction or modulation of the expression or activity of the tethered peptide. In one embodiment, the means for induction or modulation of the expression or activity of the tethered peptide comprises an inducible and/or cell type specific promoter. In a further embodiment, the means for induction or modulation of the expression or activity of the tethered peptide comprises the generation of the cassette from multiple domains or regions which must be brought together or otherwise linked for instance via protein processing, recombination or via a dimerization molecule.

The present invention also relates to a recombinant DNA molecule or cloned gene, or a degenerate variant thereof, which encodes a tethered peptide. In one such aspect, the tethered peptide is selected from a neuropeptide, neurotoxin, immune modulator, hormone or cytokine. In a particular aspect, a nucleic acid molecule, in particular a recombinant DNA molecule or cloned gene, encodes a tethering cassette comprising a promoter, a peptide to be tethered, a linker sequence, and a membrane attachment sequence. In one such aspect, the cassette further comprises a secretion signal located in the cassette prior to the peptide to be tethered. In one embodiment, the secretion signal is a signal sequence. In a further aspect, the promoter is a cell specific promoter. In an additional embodiment, the promoter is an inducible promoter. In a particular embodiment, the peptide to be tethered is selected from a neuropeptide, neurotoxin, immune modulator, hormone or cytokine.

The present invention also includes tethered peptides, which are not naturally tethered, membrane bound or membrane associated, having the activities of the natural peptide, or particularly being capable of interacting with, signaling via, or otherwise modulating at least one of their natural cell receptors or ion channels. The tethered peptides include the bungarotoxins and conotoxins specifically provided and described herein, and comprising the amino acid sequences set forth and described herein or active variants thereof.

The present invention further includes tethered peptides, which are not naturally tethered, membrane bound or membrane associated, having altered activities versus the natural peptide, or particularly being incapable of interacting with, signaling via, or otherwise modulating at least one of their natural cell receptors or ion channels—such as being capable of interacting, but not modulating or signaling via their natural cell receptors or ion channels, thus acting as antagonists to the natural peptides. The tethered peptides include mutants of neurotoxins, including the bungarotoxins and conotoxins specifically provided and described herein. These tethered peptides are exemplified by the mutant toxins provided and described herein, comprising the amino acid sequences set forth and described herein.

The tethered peptide of the present invention may be expressed in cells by introduction of a vector or DNA encoding said tethered peptide using methods and approaches known to the skilled artisan, including by transfection, infection, retrovirus infection, injection into embryos, transgenic contructs, for instance using bacterial artificial chromosomes, and by biolistic transfection or gene gun.

The present invention naturally contemplates several means for preparation of the tethered peptides, including as illustrated herein known recombinant techniques, and the invention is accordingly intended to cover such synthetic preparations within its scope. The cDNA and amino acid sequences disclosed herein and known in the art for suitable peptides, including neuropeptides and neurotoxins, facilitates the reproduction of the tethered peptides by such recombinant techniques, and accordingly, the invention extends to expression vectors prepared from the disclosed DNA or amino acid sequences, including predicted or presumed sequences, particularly in the instance of toxins which are not yet fully characterized, for expression in host systems by recombinant DNA techniques, and to the resulting transformed hosts.

The invention includes a method or assay system for screening of potential drugs effective to modulate the activity of target mammalian cells by interrupting or potentiating the tethered peptides. Thus the tethered peptides may be expressed in combination with their target receptor or ion channel and candidate modulators screened for modulation of the tethered peptide's activity, including receptor or channel activity. Alternatively, mutants or variants of the tethered peptide may be screened as candidate peptide modulators, for instance as anti-toxins in the case of tethered neurotoxins. In a further assay, one or more tethered peptide may be expressed individually or in combination with one or more candidate or orphan receptor(s), in order to identify the peptide's receptor or characterize an orphan receptor's ligand or modulating peptide. The activity of a tethered peptide may be assayed, measured or recorded by any method or means known in the art, including by channel opening or current, ion flow (including with sensitive dyes), or other method or readout.

In yet a further embodiment, the invention contemplates modulators, including agonists or antagonists of the activity of a tethered peptide, particularly including a tethered neuropeptide or toxin.

The tethered peptides, their analogs, and any antagonists or antibodies that may be raised thereto, are capable of use in connection with various diagnostic techniques, including immunoassays, such as a radioimmunoassay, using for example, the peptide or an antibody to the peptide that has been labeled by either radioactive addition, or radioiodination or which contains an epitope which is recognizable by an antibody or labeled binding agent.

For instance, in an immunoassay, a control quantity of the peptides or antibodies thereto, or the like may be prepared and labeled with an enzyme, a specific binding partner and/or a radioactive element, and may then be introduced into a cellular sample. After the labeled material or its binding partner(s) has had an opportunity to react with sites within the sample, the resulting mass may be examined by known techniques, which may vary with the nature of the label attached.

In the instance where a radioactive label, such as the isotopes ³H, ¹⁴C, ³²P, ³⁵S, ³⁶CI, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

The present invention includes an assay system which may be prepared in the form of a test kit for to identify drugs or other agents that may mimic, enhance or block the activity of the tethered peptide. The system or test kit may comprise a labeled component prepared by one of the radioactive and/or enzymatic techniques discussed herein, coupling a label to the tethered peptide, their agonists and/or antagonists or binding partners, and one or more additional immunochemical reagents, at least one of which is a free or immobilized ligand, capable either of binding with the labeled component, its binding partner, one of the components to be determined or their binding partner(s).

In a further embodiment, the present invention relates to certain therapeutic methods which would be based upon the activity of the tethered peptide(s) or upon agents or other drugs determined to possess the same activity or blocking activity. A first therapeutic method is associated with the prevention of the manifestations of conditions causally related to or following from the binding activity of the natural peptide or its subunits or the absence of or reduced expression of the natural peptide, including a neuropeptide or neurotoxin, and comprises administering the tethered peptide or an agent capable of modulating the production and/or activity of the peptide, either individually or in mixture with each other in an amount effective to prevent the development of those conditions or treat the manifestation of these conditions in the host.

It is a still further aspect of the present invention to provide a method for the treatment of mammals to control the amount or activity of a neuropeptide or toxin, so as to alter the adverse consequences of such presence or activity, or where beneficial, to enhance such activity. In one such method a tethered mutant or altered peptide neurotoxin, particularly an anti-toxin, is administered to control or treat the effects of exposure to an active neurotoxin, for example in the instance of a snake bite.

The present invention also provides a method for the treatment of mammals to control the amount or activity of a neuropeptide or toxin, so as to treat or avert the adverse consequences of invasive, spontaneous or idiopathic pathological states. In particular a method is provided for modulation of receptor, particularly ion channel, activity comprising administering, by transfection, infection, injection, transduction, etc. a tethered peptide which is capable of modulating said receptor or ion channel.

It is a still further object of the present invention to provide pharmaceutical compositions for use in therapeutic methods which comprise or are based upon the tethered peptides, their binding partner(s), or upon agents or drugs that mimic, modulate or antagonize the activities of the peptides.

Other objects and advantages will become apparent to those skilled in the art from a review of the following description which proceeds with reference to the following illustrative drawings.

### BRIEF DESCRIPTION OF THE DRAWINGS

FIG. 1A-1C depicts construction of tethered toxins for cell-autonomous inactivation of ligand and voltage gated channels. A. Model representing the binding of tethered α-bungarotoxin to the ACh binding site of the nAChR. B. Model representing the binding of tethered conotoxins to the channel pore of voltage-gated sodium and calcium channels. C. Amino acid alignments and disulfide bridges of lynxl and toxins. Tethered toxin constructs include: secretion signal sequences (grey), epitope tag (boxed in blue and placed before or after toxin sequences (DYKDDDK) (SEQ ID NO:2)), mature toxin sequences (boxed in red), synthetic polypeptide linkers of different lengths and composition (highlighted in yellow), and lynx1 hydrophobic C-terminal sequence for addition of the GPI-anchor (the very C-terminal sequences following the toxin or linker sequences (GAGFATPVTLALVPALLATFWSLLL) (SEQ ID NO:3)). Sequences of lynx1, α-Bgtx, κ-Bgtx, α-PnIB_{S}, α-MII_{S}, μO-MrVIA_{L}, and ω-MVIIA_{L} tethered toxin constructs are set out respectively in SEQ ID NOS:4-10.

FIG. 2. Tethered toxins specifically inhibit nAChRs in Xenopus oocytes. Top panel: co-expression of tethered toxins with muscle α1β1γδ nAChRs demonstrates that t-αBgtx completely blocks the muscle receptor. Middle panel: co-expression of tethered toxins with neuronal α7 nAChRs shows that t-αBgtx, t-κBgtx and t-PnIB inhibit the receptor, whereas t-MII has no effect. Bottom panel: t-KBgtx partially inhibits α4β2 nAChRs (t-test p=0.0017), while the other tethered toxins have no effect. Representative traces of ACh-evoked responses (20 s application) from individual oocytes are shown. Bar graphs represent the average +/−sem of the peak current obtained in 10 oocytes for each case.

FIG. 3. Tethered toxins specifically inhibit voltage-gated sodium and calcium channels in Xenopus oocytes. Top panel: co-expression of tethered MrVIA potently blocks the sodium Naᵥ1.2 voltage-gated channel, whereas t-MVIIA does not. Middle panel: co-expression of t-MVIIA completely blocks the calcium Caᵥ2.2 voltage-gated channel, whereas t-MrVIA has no effect. Bottom panel: potassium Shaker channels are not affected by coexpression of either t-MrVIA or t-MVIIA conotoxins. Representative traces of whole cell currents recorded from individual oocytes are shown. Voltage steps ranged from −70 to 40 mV in steps of 10 mV from a holding potential of −90 mV. A prepulse to −110 mV was done for Shaker channel recordings. Bar graphs represent the average +/−sem of the peak current obtained in 10 oocytes for each case.

FIG. 4. Tethered toxins are not cleaved from the membrane. Oocytes injected with either only α7 nAChRs or with α7 nAChRs plus tethered aBgtx were recorded to test the response to ACh. After overnight co-incubation of the two types of oocytes, electrophysiology recordings were repeated. Again 3 oocytes responded to ACh and 3 oocytes did not respond, indicating that the tethered toxin acts only on coexpresssed receptors and it does not affect neighboring cells through release from the cell surface.

FIG. 5. Functional inactivation of muscle nAChRs in vivo. A, Each field shows approximately two tail segments in intact embryos. Zebrafish embryos were injected with dual promoter constructs containing the CMV promoter upstream of EGFP and the α-αctin promoter driving expression of either t-αBgtx (left panel) or t-κBgtx (right panel). Synaptic sites on fluorescent muscle cells expressing EGFP were identified by labeling of synaptic acetylcholinesterase using FasII (second row). Labeling of AChRs with rhodamine αBgtx (R-α; third row) reveals greatly reduced levels at identified synaptic sites in the muscle cell injected with t-αBgtx. No colocalization of FasII and rhodamine αBgtx is detected in t-αBgtx injected muscle but it is prominent in t-κBgtx injected cells (bottom row). B, Labeling of nAChRs in partially dissociated muscle cells from the zebrafish mutant line twitch once that expresses receptors over the entire surface membrane. Rhodamine αBgtx labeling is effectively blocked in a single green fluorescent cell expressing t-αBgtx (left panel) and not in t-κBgtx injected cells (right panel). C, Bar graph indicating the average number +/−sem of rhodamine αBgtx labeled sites per green fluorescent muscle cell in fish injected with t-κBgtx (n=41 cells) or t-αBgtx (n=28 cells). D, Block of nAChRs in t-αBgtx injected fish assessed by electrophysiological recordings in dissociated muscle cells. The bar graph indicates the average +/−sem of ACh-evoked responses from 10 EGFP positive cells and from 10 EGFP negative cells.

FIG. 6 provides a map of a generic expression construct to clone tethered toxins with a short linker.

FIG. 7 provides a map of a generic expression construct to clone tethered toxins with a long linker.

FIG. 8 provides a map of a BAC transgenic construct for expressing tethered toxins.

FIG. 9 depicts confocal microscopy of ciliary ganglia from retrovirus infected chick embryos. Control (left panel) were injected with RCASBP vector alone and tethered α-btx1 (right panel) were injected with retrovirus RCASBP(A)-tethered-α-btx. Cells were labeled with anti-Hu C/D (Molecular Probes) or anti-α-btx and secondary antibodies.

FIG. 10 depicts epifluorescence microscopy of ciliary ganglia from retrovirus infected chick embryos. Control (upper panels) were injected with RCASBP vector alone and tethered α-btx1 (lower panels) were injected with retrovirus RCASBP(A)-tethered-α-btx. Cells were incubated with Alexa488-α-btx (Molecular Probes), washed and then photographed.

FIG. 11 provides whole cell patch clamp recordings upon incubation with the α7-nAChR specific agonist GTS-21. Recordings from two different tethered-αbtx infected neurons are shown in the two upper panels and from control vector RCASBP infected neurons in the lower panel.

FIG. 12 A and B provide (A) patch clamp recordings of oocytes coinjected with the muscle nicotinic receptor α1β1γδ alone, the receptor together with tethered-αbtx, or the receptor together with tethered mutant αbtx t-R36, and (B) graphs the average peak currents.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

The terms “tethered peptide”, “tethered neuropeptide”, “tethered neurotoxin” and “tethered toxin” and any variants not specifically listed, may be used herein interchangeably, and as used throughout the present application and claims refer to proteinaceous material and non-proteinaceous material including single or multiple proteins, particularly wherein a peptide which is not naturally tethered or attached to a cell surface or through a cell membrane is attached to or otherwise associated with a surface or membrane, particularly a cell surface or cell membrane, and extends to those proteins having the amino acid sequences described herein and presented in the FIGURES and in the TABLES, and the profile of activities set forth herein and in the claims. Accordingly, proteins displaying substantially equivalent or altered activity are likewise contemplated. These modifications may be deliberate, for example, such as modifications obtained through site-directed mutagenesis, or may be accidental, such as those obtained through mutations in hosts that are producers of the complex or its named subunits. Also, the terms “tethered peptide”, “tethered neuropeptide”, “tethered neurotoxin” and “tethered toxin” are intended to include within their scope proteins specifically recited herein as well as all substantially homologous analogs and allelic variations.

The amino acid residues described herein are preferred to be in the “L” isomeric form. However, residues in the “D” isomeric form can be substituted for any L-amino acid residue, as long as the desired fuctional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table of Correspondence:

TABLE OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more deoxyribonucleotides or ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

It should be appreciated that also within the scope of the present invention are DNA sequences encoding a tethered peptide, including those which code for a tethered peptide having the same amino acid sequence as those described herein, including in the FIGURES, but which are degenerate to such sequences. By “degenerate to” is meant that a different three-letter codon is used to specify a particular amino acid. It is well known in the art that the following codons can be used interchangeably to code for each specific amino acid:

| | |
|---|---|
| Phenylalanine (Phe or F) | UUU or UC |
| Leucine (Leu or L) | UUA or UUG or CUU or CUC |
| | or CUA or CUG |
| Isoleucine (Ile or I) | AUU or AUC or AUA |
| Methionine (Met or M) | AUG |
| Valine (Val or V) | GUU or GUC of GUA or GUG |
| Serine (Ser or S) | UCU or UCC or UCA or UCG |
| | or AGU or AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gin or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG |
| | or AGA or AGG |
| Glycine (Gly or G) | GGU or GGC or GGA or GGG |
| Tryptophan (Trp or W) | UGG |
| Termination codon | UAA (ochre) or UAG (amber) |
| | or UGA (opal) |

It should be understood that the codons specified above are for RNA sequences. The corresponding codons for DNA have a T substituted for U.

Mutations can be made in the tethered peptides of the invention such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include seguences containing conservative changes which do not significantly alter the activity or binding characteristics of the resulting protein.

The following is one example of various groupings of amino acids:

Amino Acids with Nonpolar R Groups

Alanine, Valine, Leucine, Isoleucine, Proline, Phenylalanine, Tryptophan, Methionine
   Amino Acids with Uncharged Polar R Groups
Glycine, Serine, Threonine, Cysteine, Tyrosine, Asparagine, Glutamine
   Amino Acids with Charged Polar R Groups (Negatively Charged at Ph 6.0)
Aspartic acid, Glutamic acid
   Basic Amino Acids (Positively Charged at pH 6.0)
Lysine, Arginine, Histidine (at pH 6.0)
   Another grouping may be those amino acids with phenyl groups:
Phenylalanine, Tryptophan, Tyrosine

Another grouping may be according to molecular weight (i.e., size of R groups):

| | |
|---|---|
| Glycine | 75 |
| Alanine | 89 |
| Serine | 105 |
| Proline | 115 |
| Valine | 117 |
| Threonine | 119 |
| Cysteine | 121 |
| Leucine | 131 |
| Isoleucine | 131 |
| Asparagine | 132 |
| Aspartic acid | 133 |
| Glutamine | 146 |
| Lysine | 146 |
| Glutamic acid | 147 |
| Methionine | 149 |
| Histidine (at pH 6.0) | 155 |
| Phenylalanine | 165 |
| Arginine | 174 |
| Tyrosine | 181 |
| Tryptophan | 204 |

Particularly preferred substitutions are:
Lys for Arg and vice versa such that a positive charge may be maintained;
Glu for Asp and vice versa such that a negative charge may be maintained;
Ser for Thr such that a free —OH can be maintained; and
Gln for Asn such that a free NH₂ can be maintained.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

Mutations or alterations in the tethered peptides of the present invention further include and comprise peptides having one or more amino acid deletion. Any such deletion may have no significant effect on the peptide's activity, or may alter the activity such that the tethered peptide lacks modulating or signaling activity of the natural peptides. In one such instance, the altered, inactive peptide ha antagonist activity, blocking the natural peptide from modulating or signaling but failing to modulate or signal itself, such as an anti-toxin.

Two amino acid sequences are “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F(ab)₂ and F(v), which portions are preferred for use in the therapeutic methods described herein. Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to prevent, and preferably reduce by at least about 30 percent, more preferably by at least 50 percent, most preferably by at least 90 percent, a clinically significant change in the S phase activity of a target cellular mass, or other feature of pathology such as for example, elevated blood pressure, fever or white cell count as may attend its presence and activity.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20NC below the predicted or determined Tₘ with washes of higher stringency, if desired.

In its broadest aspect, the present invention extends to a method for tethering peptides, particularly peptides which are not naturally tethered or attached to a cell surface or through a cell membrane, including naturally occurring and mutant or altered peptide sequences, to a surface, including the cell surface. In a particular aspect, these peptides interact with, signal via, or otherwise modulate cell receptors or ion channels.

In accordance with the present invention, a method is provided for tethering neuropeptides and peptide toxins, including naturally occurring and mutant or altered neuropeptides and toxins, to a surface, including the cell surface. In a particular aspect, these neuropeptides and toxins interact with, signal via, or otherwise modulate cell receptors or ion channels, particularly including neural cell receptors or ion channels, including for instance acetylcholine (nicotinic) receptors, glutamate receptors, serotonin receptors, GABA receptors, calcium channels, sodium channels and potassium channels.

In accordance with the present invention, a method is provided for tethering immune modulators, including naturally occurring and mutant or altered immune modulators, to a surface, including the cell surface. In a particular aspect, these immune modulators interact with, signal via, or otherwise modulate cell receptors or ion channels involved in the immune system or immune response, particularly including T cell receptors.

In accordance with the present invention, a method is provided for tethering cytokines, including naturally occurring and mutant or altered cytokines, to a surface, including the cell surface. In a particular aspect, these cytokines interact with, signal via, or otherwise modulate cell receptors or ion channels, particularly to generate an intracellular signal that modulates or alters cell growth or response, including in the hematopoietic system.

In accordance with the present invention, a method is provided for tethering hormones, including naturally occurring and mutant or altered hormones, to a surface, including the cell surface. In a particular aspect, these hormones interact with, signal via, or otherwise modulate cell receptors or ion channels, including for example leptin, which interacts with the leptin receptor DB.

In an aspect of the invention, the peptide, neuropeptide or toxin acts on a receptor or ion channel at the cell membrane and is expressed in tethered form in and on the cell expressing its receptor or ion channel. Thus, the tethered peptide acts in a cell autonomous fashion, modulating or signaling via its receptor or ion channel without the necessary addition of other or other types of cells.

In a further aspect of the invention, the peptide, neuropeptide or toxin acts on a receptor or ion channel at the cell membrane and is expressed in tethered form in and on a distinct cell, not expressing its receptor or ion channel. Thus, the tethered peptide acts in a cell dependent fashion, in trans, for instance acting on a synaptic partner, modulating or signaling via its receptor or ion channel upon the addition of other or other types of cells expressing its receptor or ion channel.

In one aspect, a neuroactive toxin, particularly selected from a spider, snake, scorpion, snail, bacteria, bee or fish toxin is tethered to the surface of a cell, particularly a neural cell or cell involved with or associated with the nervous system or nerve cell response.

The invention provides a tethered peptide attached to or integrated in a cell membrane, cell or other surface, which is not naturally tethered, membrane bound or membrane associated. The invention further provides a tethered neuropeptide or toxin which is attached to or integrated in a cell membrane, cell or other surface, which is not naturally tethered, membrane bound or membrane associated. The invention particularly provides a tethered conotoxin or bungarotoxin. In a further particular aspect, a tethered ω-o-conotoxin, α-bungarotoxin or κ-bungarotoxin is provided.

The neuropeptides and toxins may be tethered or otherwise attached to a cell membrane by various means, including utilizing known and natural sequences from membrane and/or cell surface proteins which are naturally attached to or transverse through the cell membrane, or any such other means known or available in the art to attach or associate a peptide or molecule with the membrane or cell surface, including via attachment to or association with a membrane protein. Sequences from known and natural membrane and/or cell surface proteins serve to transverse or attach to the membrane per se or are recognized and function to signal attachment and/or specific modification in cells. The peptides of the present invention may be tethered in accordance with the invention via a membrane attachment sequence. A membrane attachment sequence may be selected from a transmembrane domain, a hydrophobic domain, a GPI attachment sequence, a myristoylation sequence, a palmitoylation sequence (Cys-A-A-X) (SEQ ID NO:1), or any other such peptide sequence which encodes for or signals the attachment of a lipid or hydrophobic sequence or the association of a peptide with the membrane.

Thus, the invention includes a tethering cassette for expression of or preparation of a tethered peptide, comprising a promoter, a peptide to be tethered, a linker sequence and a membrane attachment sequence. In a particular embodiment, the cassette additionally comprises a secretion signal located in the cassette prior to the peptide to be tethered. In one embodiment, the secretion signal is a signal sequence. In one embodiment, the promoter is a cell specific promoter. In a further embodiment, the promoter is an inducible promoter. In a particular embodiment, the peptide to be tethered is selected from a neuropeptide, neurotoxin, immune modulator, hormone or cytokine.

The invention provides a tethering system for expression of or preparation of a tethered peptide, comprising a promoter, a peptide to be tethered, a linker sequence and a membrane attachment sequence, and further comprising a means for induction or modulation of the expression or activity of the tethered peptide. In one embodiment, the means for induction or modulation of the expression or activity of the tethered peptide comprises an inducible and/or cell type specific promoter. In a further embodiment, the means for induction or modulation of the expression or activity of the tethered peptide comprises the generation of the cassette from multiple domains or regions which must be brought together or otherwise linked for instance via protein processing, recombination or via a dimerization molecule.

Approaches and methods suitable as a means for induction or modulation of the expression or activity of the tethered peptide are known and may be selected as suitable or applicable by the skilled artisan. Exemplary such approaches and methods include an inducible promoter system, for instance the tet system, and recombination based activation or expression, for instance, the cre-lox system (Kilby, N et al (1993) Trends Genet 9:413-421; U.S. Pat. No. 5,658,772). Tetracysteinespeptide motifs may be utilized with biarsenical ligands, including the FlAsH system (Thorn K S et al Prot Sci (2000) 9(2):213-7; Griffin B A et al (2000) Meth Enzym 327:565-78; Adams S R et al J Am Chem Soc (2002) 124(21):6063-76; Sosinsky G E et al (2003) Cell Commun Adhes 10(4-6):181-6; Kapahi P et al (2000) J Biol Chem 275(46):36062-6). Conditional protein splicing, or expressed protein ligation, in which an intervening intein domain excises autocatalytically from a precursor polypeptide, linking the flanking extein sequences is also contemplated (Mootz H D et al (2003) J Am Chem Soc 125(35): 10561-9; Muir T W (2003) Ann Rev Biochem 72:249-89; Severinov K and Muir T W (1998) J Biol Chem 273(26): 16205-9).

The present invention also relates to a recombinant DNA molecule or cloned gene, or a degenerate variant thereof, which encodes a tethered peptide. In one such aspect, the tethered peptide is selected from a neuropeptide, neurotoxin, immune modulator, hormone or cytokine. In a particular aspect, a nucleic acid molecule, in particular a recombinant DNA molecule or cloned gene, encodes a tethering cassette comprising a promoter, a peptide to be tethered, a linker sequence, and a membrane attachment sequence. In one such aspect, the cassette further comprises a secretion signal located in the cassette prior to the peptide to be tethered. In one embodiment, the secretion signal is a signal sequence. In a further aspect, the promoter is a cell specific promoter. In an additional embodiment, the promoter is an inducible promoter. In a particular embodiment, the peptide to be tethered is selected from a neuropeptide, neurotoxin, immune modulator, hormone or cytokine.

The present invention also includes tethered peptides, which are not naturally tethered, membrane bound or membrane associated, having the activities of the natural peptide, or particularly being capable of interacting with, signaling via, or otherwise modulating at least one of their natural cell receptors or ion channels. The tethered peptides include the bungarotoxins and conotoxins specifically provided and described herein, and comprising the amino acid sequences set forth and described herein or active variants thereof.

The present invention further includes tethered peptides, which are not naturally tethered, membrane bound or membrane associated, having altered activities versus the natural peptide, or particularly being incapable of interacting with, signaling via, or otherwise modulating at least one of their natural cell receptors or ion channels—such as being capable of interacting, but not modulating or signaling via their natural cell receptors or ion channels, thus acting as antagonists to the natural peptides (for instance anti-toxins). The tethered peptides include mutants of neurotoxins, including the bungarotoxins and conotoxins specifically provided and described herein. These tethered peptides are exemplified by the mutant toxins provided and described herein, comprising the amino acid sequences set forth and described herein.

The tethered peptide of the present invention may be expressed in cells by introduction of a vector or DNA encoding said tethered peptide using methods and approaches known to the skilled artisan, including by transfection, infection, retrovirus infection, injection into embryos, transgenic contructs, for instance using bacterial artificial chromosomes, and by biolistic transfection or gene gun.

The present invention naturally contemplates several means for preparation of the tethered peptides, including as illustrated herein known recombinant techniques, and the invention is accordingly intended to cover such synthetic preparations within its scope. The cDNA and amino acid sequences disclosed herein and known in the art for suitable peptides, including neuropeptides and neurotoxins, facilitates the reproduction of the tethered peptides by such recombinant techniques, and accordingly, the invention extends to expression vectors prepared from the disclosed DNA or amino acid sequences, including predicted or presumed sequences, particularly in the instance of toxins which are not yet fully characterized, for expression in host systems by recombinant DNA techniques, and to the resulting transformed hosts.

The invention includes a method or assay system for screening of potential drugs effective to modulate the activity of target mammalian cells by interrupting or potentiating the tethered peptides. Thus the tethered peptides may be expressed in combination with their target receptor or ion channel and candidate modulators screened for modulation of the tethered peptide's activity, including receptor or channel activity. Alternatively, mutants or variants of the tethered peptide may be screened as candidate peptide modulators, for instance as anti-toxins in the case of tethered neurotoxins. In a further assay, one or more tethered peptide may be expressed individually or in combination with one or more candidate or orphan receptor(s), in order to identify the peptide's receptor or characterize an orphan receptor's ligand or modulating peptide. The activity of a tethered peptide may be assayed, measured or recorded by any method or means known in the art, including by channel opening or current, ion flow (including with sensitive dyes), or other method or readout. Methods for measuring receptor activity and response are well known and familiar to those of skill in the art and include the use of voltage clamp assays, oocyte assays, and indicator dyes which are sensitive to or record receptor activity and/or changes in intracellular ion concentration.

In yet a further embodiment, the invention contemplates modulators, including agonists or antagonists of the activity of a tethered peptide, particularly including a tethered neuropeptide or toxin.

The tethered peptides, their analogs, and any antagonists or antibodies that may be raised thereto, are capable of use in connection with various diagnostic techniques, including immunoassays, such as a radioimmunoassay, using for example, the peptide or an antibody to the peptide that has been labeled by either radioactive addition, or radioiodination or which contains an epitope which is recognizable by an antibody or labeled binding agent.

For instance, in an immunoassay, a control quantity of the peptides or antibodies thereto, or the like may be prepared and labeled with an enzyme, a specific binding partner and/or a radioactive element, and may then be introduced into a cellular sample. After the labeled material or its binding partner(s) has had an opportunity to react with sites within the sample, the resulting mass may be examined by known techniques, which may vary with the nature of the label attached.

In the instance where a radioactive label, such as the isotopes ³H, ¹⁴C, ³²P, ³⁵S, ³⁶Cl, ⁵¹Cr, ⁵⁷Co, ⁵⁸Co, ⁵¹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

The present invention includes an assay system which may be prepared in the form of a test kit for to identify drugs or other agents that may mimic, enhance or block the activity of the tethered peptide. The system or test kit may comprise a labeled component prepared by one of the radioactive and/or enzymatic techniques discussed herein, coupling a label to the tethered peptide, their agonists and/or antagonists or binding partners, and one or more additional immunochemical reagents, at least one of which is a free or immobilized ligand, capable either of binding with the labeled component, its binding partner, one of the components to be determined or their binding partner(s).

In a further embodiment, the present invention relates to certain therapeutic methods which would be based upon the activity of the tethered peptide(s) or upon agents or other drugs determined to possess the same activity or blocking activity. A first therapeutic method is associated with the prevention of the manifestations of conditions causally related to or following from the binding activity of the natural peptide or its subunits or the absence of or reduced expression of the natural peptide, including a neuropeptide or neurotoxin, and comprises administering the tethered peptide or an agent capable of modulating the production and/or activity of the peptide, either individually or in mixture with each other in an amount effective to prevent the development of those conditions or treat the manifestation of these conditions in the host.

It is a still further aspect of the present invention to provide a method for the treatment of mammals to control the amount or activity of a neuropeptide or toxin, so as to alter the adverse consequences of such presence or activity, or where beneficial, to enhance such activity. In one such method a tethered mutant or altered peptide neurotoxin, particularly an anti-toxin, is administered to control or treat the effects of exprosure to an active neurotoxin, for example in the instance of a snake bite.

The present invention also provides a method for the treatment of mammals to control the amount or activity of a neuropeptide or toxin, so as to treat or avert the adverse consequences of invasive, spontaneous or idiopathic pathological states. In particular a method is provided for modulation of receptor, particularly ion channel, activity comprising administering, by transfection, infection, injection, transduction, etc. a tethered peptide which is capable of modulating said receptor or ion channel.

It is a still further object of the present invention to provide pharmaceutical compositions for use in therapeutic methods which comprise or are based upon the tethered peptides, their binding partner(s), or upon agents or drugs that mimic, modulate or antagonize the activities of the peptides.

### Tetherine to Cell Membranes

The neuropeptides and toxins may be tethered or otherwise attached to a cell membrane by various means, including utilizing known and natural sequences from membrane and/or cell surface proteins which are naturally attached to or transverse through the cell membrane, or any such other means known or available in the art to attach or associate a peptide or molecule with the membrane or cell surface, including via attachment to or association with a membrane protein. Sequences from known and natural membrane and/or cell surface proteins serve to transverse or attach to the membrane per se or are recognized and function to signal attachment and/or specific modification in cells. Such sequences will be known and recognized to the skilled artisan and include for instance such sequences or domains as transmembrane domains, PH domains, GPI-anchor sequences, palmitoylation sites (Cys-A-A-X) (SEQ ID NO:1), and myristoylation sites. In addition, the peptides may be tethered via association with a membrane protein, for instance, by possessing a sequence which binds to a membrane receptor, or a sequence or domain which associates by dimerization or multimerization with a membrane bound or membrane associating sequence, or by attachment of an antigen or antibody and association or interaction with the membrane or a membrane protein therewith or therethrough.

Cell membranes contain at least 25% proteins, membrane proteins, which are active components of membranes for transport, signaling, and cell-cell communication (for instance, receptors adhesion molecules). Many membrane proteins are transmembrane proteins having functional domains on either side of a membrane and a hydrophobic transmembrane domain amino acid sequence through the membrane. Membrane proteins can be attached to the membrane surface through lipid anchors or electrostatic binding. Lipid anchors are fatty acids or isoprenoids (geranyl, farnesyl) which are covalently linked to amino acids and provide a close attachment yet lateral mobility along the membrane surface. Lipid anchors include palmitoylation, myristoylation and GPI anchors. Palmitoylation is acquired post-translationally and cytoplasmically and not in the ER. A common recognition site for palmitoylation is Cys-A-A-X, with A denoting aliphatic amino acid and X any C-terminal amino acid. Myristoylation is coupled to protein translation, with co-translational modification at the N-terminus by N-myristoyltransferase (NMT). An N-terminal glycine, followed by a either a Asn, Gln, Ser, Val or Leu (and not a Asp, D-Asn, Phe or Tyr) is recognized by the NMT.

Many eukaryotic cell surface proteins are anchored to the plasma membrane via glycosylphosphatidylinositol (GPI). The GPI transamidase mediates GPI anchoring in the endoplasmic reticulum by replacing a protein's C-terminal GPI attachment signal peptide with a pre-assembled GPI. GPI is widely utilized in unicellular and higher eukaryotes (McConville M J and Ferguson M A (1993) Biochem J 294(Pt2):305-24). In eukaryotes, GPI-linked proteins include: antigens and lymphocyte surface proteins, including carcioembryonic antigen, Thy-1, Ly-6, CD16, CD55, CD59 and Qa-2; cell surface hydrolases, including alkaline phosphatase, acetylcholinesterase (AchE), and 5′nucleotidase; adhesion molecules, including neural cell adhesion molecule and heparin sulfate proteoglycan; semaphorins; ephrin-A ligands (including B61, AL-1/RAGS, LERK4 and ELF-1) and the neural protein Lynx1. Protozoal antigens and other proteins which are GPI-anchored include trypanosome VSG, leishmanial protease, plasmodium antigens, scrapie prion protein, folate receptor and human erythrocyte decay accelerating factor (DAF). Eisenhaber et al have investigated and reported on post-translational GPI lipid anchor modification of proteins in kingdoms of life, providing an analysis of protein sequence data from complete genomes, finding GPI modification in approximately 0.5% of all proteins among lower and higher Eukaryota (Eisenhaber B et al (2001) Protein Eng 14(1): 17-25). Their list of potentially modified proteins with their predicted cleavage sites, as well as access to the GPI predictor software tool, big-pi, is available at mendel.imp.univie.ac.at/gpi/gpi_genomes.html.

The consensus sequence rules for C-terminal sequences signaling the addition of GPI anchors include: (i) residue to which anchor is attached (termed ω site) and residue two amino acids on carboxyl side (ω+2 site) have small side chains (for instance glycine, alanine, serine, threonine); (ii) the ω+1 site amino acid can have large side chains; (iii) the ω+2 site is followed by 5 to 10 hydrophilic amino acids; (iv) then 15-20 hydrophobic amino acids follow at or near the carboxy terminus. Examples of C-terminal sequences signaling the addition of GPI anchors are provided in TABLE 2. The large bold amino acid in each TABLE 2 sequence is the site of GPI attachment. The bold sequence present after or to the right of the large bold amino acid is cleaved by the transpeptidase upon GPI anchor addition.

TABLE 2

Examples of C-Terminal Sequences
Signaling The Addition of GPI-Anchors

| | |
|---|---|
| PROTEIN | GPI-SIGNAL SEQUENCE |

| | | |
|---|---|---|
| Acetylcholinesterase | NQFLPKLLNATA [symbol] | (SEQ ID NO:11) |
| (Torpedo) | DGELSSSGIIFYVLYLIFY | |
| Alkaline Phosphatase | TACDLAPPAGTT [symbol] | (SEQ ID NO:12) |
| (placenta) | AAHPGRSVVPALLPLLAGTLLLLETATAP | |
| Decay Accelerating | HETTPNKGSGTT [symbol] | (SEQ ID NO:13) |
| Factor | GTTRLLSGHTCFLTTGLLGTLVTMGLLT | |
| PARP (T. Brucei) | EPEPEPEPEPEP [symbol] | (SEQ ID NO:14) |
| | AATLKSVALPFAIAAAALVAAF | |
| Prion Protein | QKESQAYYDGRR [symbol] | (SEQ ID NO:15) |
| (hamster) | SAVLFSSPPVILLISFLIFLMVG | |
| Thy-1 (rat) | KTINVIRDKLVK [symbol] | (SEQ ID NO:16) |
| | GGISLLVQNTSWLLLLLLSLSFLQATDFISI | |
| Variant Surface | ESNCKWENNACK [symbol] | (SEQ ID NO:17) |
| Glycoprotein | SSILVTKKFALTVVSAAFVALLF | |
| (T.Brucei) | | |

### Tethering to Surfaces

It is contemplated in the present invention that the neuropeptides and/or toxins may be tethered or attached to a surface, including a biological surface, a biomaterial, a membrane, a polymeric carrier(s), biodegradable or biomimetic matrices or a scaffold. Examples of such surfaces, materials or membranes include, but are not limited to polyglycolic acid (PGA), polylactic acid (PLA), hyaluronic acid, gelatin, cellulose, nitrocellulose, PVDF, collagen, albumin, fibrin, alginate, cotton, nylon (polyamides), dacron (polyesters), polystyrene, polypropylene, polyacrylates, polyvinyl compounds (e.g., polyvinylchloride), polycarbonate (PVC), polytetrafluorethylene (PTFE, teflon), thermanox (TPX), polymers of hydroxy acids such as polylactic acid (PLA), polyglycolic acid (PGA), and polylactic acid-glycolic acid (PLGA), polyorthoesters, polyanhydrides, polyphosphazenes, and a variety of polyhydroxyalkanoates, and combinations thereof.

Antibodies to the tethered peptide or to a component thereof, including both polyclonal and monoclonal antibodies, and drugs that modulate the production or activity of the peptide and/or their subunits may possess certain diagnostic applications and may for example, be utilized for the purpose of detecting and/or measuring conditions such as toxin exposure, toxin levels, bacterial infection, viral infection or the like. For example, the tethered peptide or its subunits may be used to produce both polyclonal and monoclonal antibodies to themselves in a variety of cellular media, by known techniques such as the hybridoma technique utilizing, for example, fused mouse spleen lymphocytes and myeloma cells. Likewise, small molecules that mimic or antagonize the activity(ies) of the tethered peptide of the invention may be discovered or synthesized, and may be used in diagnostic and/or therapeutic protocols.

The general methodology for making monoclonal antibodies by hybridomas is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890.

Panels of monoclonal antibodies produced against the peptides of the invention can be screened for various properties; i.e., isotype, epitope, affinity, etc. Of interest are monoclonal antibodies that neutralize the activity of the peptide or its subunits. Such monoclonals can be readily identified in peptide activity assays. High affinity antibodies are also useful when immunoaffinity purification of native or recombinant peptide is desired. Particularly, the anti-peptide antibody used in the diagnostic methods of this invention is an affinity purified polyclonal antibody. More particularly, the antibody is a monoclonal antibody (mAb). In addition, the anti-peptide antibody molecules used herein may be in the form of Fab, Fab′, F(ab′)₂ or F(v) portions of whole antibody molecules.

Methods for producing polyclonal anti-polypeptide antibodies are well-known in the art. See U.S. Pat. No. 4,493,795 to Nestor et al. A monoclonal antibody, typically containing Fab and/or F(ab′)₂ portions of useful antibody molecules, can be prepared using the hybridoma technology described in Antibodies—A Laboratory Manual, Harlow and Lane, eds., Cold Spring Harbor Laboratory, New York (1988), which is incorporated herein by reference. Briefly, to form the hybridoma from which the monoclonal antibody composition is produced, a myeloma or other self-perpetuating cell line is fused with lymphocytes obtained from the spleen of a mammal hyperimmunized with a peptide-binding portion thereof, or peptide, or an origin-specific DNA-binding portion thereof. Niman et al., Proc. Natl. Acad. Sci. USA, 80:4949-4953 (1983) also provide methods for producing monoclonal anti-peptide antibodies. Typically, the peptide or a peptide analog is used either alone or conjugated to an immunogenic carrier and the hybridomas are screened for the ability to produce an antibody that immunoreacts with the peptide or peptide analog.

A monoclonal antibody useful in practicing the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques. Media useful for the preparation of these compositions are both well-known in the art and commercially available and include synthetic culture media, inbred mice and the like. An exemplary synthetic medium is Dulbecco's minimal essential medium (DMEM; Dulbecco et al., Virol. 8:396 (1959)) supplemented with 4.5 gm/l glucose, 20 mm glutamine, and 20% fetal calf serum. An exemplary inbred mouse strain is the Balb/c.

As discussed earlier, the tethered peptides or their binding partners or other ligands or agents exhibiting either mimicry or antagonism to the peptides or control over their production, may be prepared in pharmaceutical compositions, with a suitable carrier and at a strength effective for administration by various means to a patient experiencing an adverse medical condition associated with the peptide, in the case of a toxin, or with modulation of its target receptor or ion channel, for the treatment thereof. A variety of administrative techniques may be utilized, among them parenteral techniques such as subcutaneous, intravenous and intraperitoneal injections, catheterizations and the like.

Average quantities of the peptide or their subunits may vary and in particular should be based upon the recommendations and prescription of a qualified physician or veterinarian.

The present invention further contemplates therapeutic compositions useful in practicing the therapeutic methods of this invention. A subject therapeutic composition includes, in admixture, a pharmaceutically acceptable excipient (carrier) and one or more of a tethered peptide, analog thereof or fragment thereof, as described herein as an active ingredient. In one embodiment, the composition comprises an agent capable of modulating the specific binding of the present tethered peptide within a target cell. The preparation of therapeutic compositions which contain polypeptides, analogs or active fragments as active ingredients is well understood in the art. Such compositions may be prepared as injectables, either as liquid solutions or suspensions, and solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A polypeptide, analog or active fragment can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic polypeptide-, analog- or active fragment-containing compositions are conventionally administered intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of inhibition or neutralization of peptide binding capacity desired. Precise amounts of active ingredient required to be administered depend on the judgment of the practitioner and are peculiar to each individual. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

The therapeutic compositions may further include an effective amount of the tethered peptide, antagonist or analog thereof, and one or more of the following active ingredients: a neuropeptide, a neuromodulatory molecule, an immune modulator, a neuroactive agent or drug, a steroid.

As used herein, “pg” means picogram, “ng” means nanogram, “ug” or “μg” mean microgram, “mg” means milligram, “ul” or “μl” mean microliter, “ml” means milliliter, “1” means liter.

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host.

Such operative linking of a DNA sequence of this invention to an expression control sequence, of course, includes, if not already part of the DNA sequence, the provision of an initiation codon, ATG, in the correct reading frame upstream of the DNA sequence.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products.

Considering these and other factors a person skilled in the art will be able to construct a variety of vector/expression control sequence/host combinations that will express the DNA sequences of this invention on fermentation or in large scale animal culture.

It is further intended that tethered peptide analogs may be prepared from nucleotide sequences of the protein complex/subunit derived within the scope of the present invention. Analogs, such as fragments, may be produced, for example, by protease, e.g. pepsin digestion of the peptide. Other analogs, such as muteins, can be produced by standard site-directed mutagenesis of peptide coding sequences. Analogs exhibiting “peptide activity” such as small molecules, whether functioning as promoters or inhibitors, may be identified by known in vivo and/or in vitro assays.

As mentioned above, a DNA sequence encoding the peptide to be tethered can be prepared synthetically rather than cloned. The DNA sequence can be designed with the appropriate codons for the peptide amino acid sequence. In general, one will select preferred codons for the intended host if the sequence will be used for expression. The complete sequence is assembled from overlapping oligonucleotides prepared by standard methods and assembled into a complete coding sequence. See, e.g., Edge, Nature, 292:756 (1981); Nambair et al., Science, 223:1299 (1984); Jay et al., J. Biol. Chem., 259:6311 (1984).

Synthetic DNA sequences allow convenient construction of genes which will express peptide analogs or “muteins”. Alternatively, DNA encoding muteins can be made by site-directed mutagenesis of native peptide genes or cDNAs or by synthesis of short amino acids, particularly in the instance or small neurotoxins, and muteins can be made directly using conventional polypeptide synthesis.

Additionally, the peptide to be tethered can be prepared synthetically rather than cloned. Methods for chemical synthesis of peptides are well known in the art.

A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). This method may be used to create analogs with unnatural amino acids.

The tethered peptide(s) of the invention may be labeled. The labels most commonly employed are radioactive elements, enzymes, chemicals which fluoresce when exposed to ultraviolet light, and others. A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate. The tethered peptide or its binding partner(s) can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re.

Enzyme labels are likewise useful, and can be detected by any of the presently utilized calorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

A particular assay system developed and utilized in accordance with the present invention, is known as a receptor assay. In a receptor assay, the material to be assayed is appropriately labeled and then certain cellular test colonies are inoculated with a quantity of both the labeled and unlabeled material after which binding studies are conducted to determine the extent to which the labeled material binds to the cell receptors. In this way, differences in affinity between materials can be ascertained.

Accordingly, a purified quantity of the tethered peptide may be radiolabeled and combined, for example, with antibodies or other inhibitors thereto, after which binding studies would be carried out. Solutions would then be prepared that contain various quantities of labeled and unlabeled uncombined peptide, and cell samples would then be inoculated and thereafter incubated. The resulting cell monolayers are then washed, solubilized and then counted in a gamma counter for a length of time sufficient to yield a standard error of <5%. These data are then subjected to Scatchard analysis after which observations and conclusions regarding material activity can be drawn. While the foregoing is exemplary, it illustrates the manner in which a receptor assay may be performed and utilized, in the instance where the cellular binding ability of the assayed material may serve as a distinguishing characteristic.

An assay useful and contemplated in accordance with the present invention is known as a “cis/trans” assay. Briefly, this assay employs two genetic constructs, one of which is typically a plasmid that continually expresses a particular receptor of interest when transfected into an appropriate cell line, and the second of which is a plasmid that expresses a reporter such as luciferase, under the control of a receptor/ligand complex. Thus, for example, if it is desired to evaluate a compound as a ligand for a particular receptor, one of the plasmids would be a construct that results in expression of the receptor in the chosen cell line, while the second plasmid would possess a promoter linked to the luciferase gene in which the response element to the particular receptor is inserted. If the compound under test is an agonist for the receptor, the ligand will complex with the receptor, and the resulting complex will bind the response element and initiate transcription of the luciferase gene. The resulting chemiluminescence is then measured photometrically, and dose response curves are obtained and compared to those of known ligands. The foregoing protocol is described in detail in U.S. Pat. No. 4,981,784 and PCT International Publication No. WO 88/03168, for which purpose the artisan is referred.

In a further embodiment of this invention, commercial test kits suitable for use by a medical specialist may be prepared to determine the presence or absence of predetermined peptide activity or predetermined peptide target receptor or ion channel activity in suspected target cells. In accordance with the testing techniques discussed above, one class of such kits will contain at least the labeled peptide or its binding partner, for instance an antibody specific thereto, and directions, of course, depending upon the method selected, e.g., “competitive,” “sandwich,” “DASP” and the like. The kits may also contain peripheral reagents such as buffers, stabilizers, etc.

Accordingly, a test kit may be prepared for the demonstration of the presence or capability of cells for predetermined peptide activity, comprising:
(a) a predetermined amount of at least one labeled immunochemically reactive component obtained by the direct or indirect attachment of the peptide or a specific binding partner thereto, to a detectable label;
(b) other reagents; and
(c) directions for use of said kit.

More specifically, the diagnostic test kit may comprise:
(a) a known amount of the peptide as described above (or a binding partner) generally bound to a solid phase to form an immunosorbent, or in the alternative, bound to a suitable tag, or plural such end products, etc. (or their binding partners) one of each;
(b) if necessary, other reagents; and
(c) directions for use of said test kit.
   In a further variation, the test kit may be prepared and used for the purposes stated above, which operates according to a predetermined protocol (e.g. “competitive,” “sandwich,” “double antibody,” etc.), and comprises:
(a) a labeled component which has been obtained by coupling the peptide to a detectable label;
(b) one or more additional immunochemical reagents of which at least one reagent is a ligand or an immobilized ligand, which ligand is selected from the group consisting of:
   (i) a ligand capable of binding with the labeled component (a);
   (ii) a ligand capable of binding with a binding partner of the labeled component (a);
   (iii) a ligand capable of binding with at least one of the component(s) to be determined; and
   (iv) a ligand capable of binding with at least one of the binding partners of at least one of the component(s) to be determined; and
(c) directions for the performance of a protocol for the detection and/or determination of one or more components of an immunochemical reaction between the peptide and a specific binding partner thereto.

In accordance with the above, an assay system for screening potential drugs effective to modulate the activity of the tethered peptide may be prepared. The peptide may be introduced into a test system, and the prospective drug may also be introduced into the resulting cell culture, and the culture thereafter examined to observe any changes in the peptide activity of the cells, due either to the addition of the prospective drug alone, or due to the effect of added quantities of the known peptide.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLE 1

### Tethering Naturally Occurring Peptide Toxins for Cell Autonomous Modulation of Ion Channels and Receptors In Vivo

The physiologies of cells, and their roles in complex multi-cellular organisms, depend on the electrical and chemical signals carried by a broad array of ion channels and receptors. Venomous animals, including elapid snakes, cone snails and spiders, produce an enormous variety of peptide toxins that exert their toxic action by binding with high affinity to specific ion channels and receptors. We previously identified a mammalian prototoxin, lynxl, which shares with α-bungarotoxin the ability to bind and modulate nicotinic acetylcholine receptors (nAChRs). Unlike venom toxins, lynx1 is tethered to the cell surface via a glycophospholipid (GPI) anchor. We show here that several classes of neurotoxins, including bungarotoxins and conotoxins, retain their selective antagonistic properties when tethered to the cell surface. Further, expression of tethered α-bungarotoxin in developing zebrafish results in targeted elimination of nAChR function in vivo, thus silencing synaptic transmission without interfering with synapse formation. These studies harness the pharmacological properties of peptide toxins for use in genetic experiments. When combined with methods to achieve cell and temporal specificity of expression, the extension of this approach to the hundreds of naturally occurring peptide toxins opens a new landscape for cell autonomous regulation of cellular physiology in vivo.

Introduction

Our current understanding of the development and function of the mammalian brain rests in large part on conclusions from experimental or accidental lesions that perturb CNS function. These studies have involved a wide variety of techniques, including analysis of behavioral deficits in people suffering from disease or acute brain injury (Manns et al, 2003), sensory deprivation during development of experimental animals (Shatz and Stryker, 1978), pharmacologic manipulations of CNS receptors and ion channels (Bauer et al, 2002; Shatz and Strykerl988), and genetic ablations of specific CNS cell types in experimental animals (Nakazawa et al, 2004, Kofuji et al, 2000, Champtiaux and Changeux 2004). This rich history, and advances in genetic techniques that enable in vivo manipulations of molecules, cells and circuits (KOs, BACs, siRNA), provide a powerful incentive for development of genetic approaches that extend our ability to alter the physiologic properties of specific neurons in vivo. For example, the ability to block pre-synaptic vesicle fusion using botulinum neurotoxins has been introduced as an effective approach to genetically interfere with neurotransmission in specific CNS cell types (Steinhardt et al, 1994; Saint-Amant and Drapeau 2001). Likewise, targeted overexpression of K⁺ channels has been shown to be highly effective at silencing neuronal activity both in mammalian and Drosophila excitable cells (Johns et al. 1999; Nadeau et al. 2000; Paradis et al. 2001; Nitabach, et al, 2002), although in mammalian cells this approach can induce apoptotic cell death in neurons (Nadeau et al., 2000).

Based on knowledge that the electrical properties of neurons are controlled by a remarkable variety of ionic currents carried by many specific ion channels and receptors (Champtiaux and Changeux 2004, Caterall 1999, Olivera 1994, Coetzee et al, 1999), and the fact that small specifically acting soluble peptide neurotoxins have evolved specifically to block many of these currents (Adams and Olivera 1994, Terlau and Olivera 2004, Olivera et al, 1994, Tsetlin and Hucho 2004, McIntosh et al, 1999), we sought to harness the potential of peptide toxins as a means to genetically manipulate the functional properties of CNS neurons. To do this, we generated fusion proteins based on the lynx1 prototoxin (Miwa et al, 1999; Ibanez-Tallon et al, 2002), but carrying functional domains from naturally occurring peptide neurotoxins. These chimeric toxins are tethered to the cell surface via a GPI anchor, yet retain their ability to specifically block ligand and voltage gated receptors and ion channels cell autonomously in Xenopus oocytes and in vivo in zebrafish muscle fibers.

Results

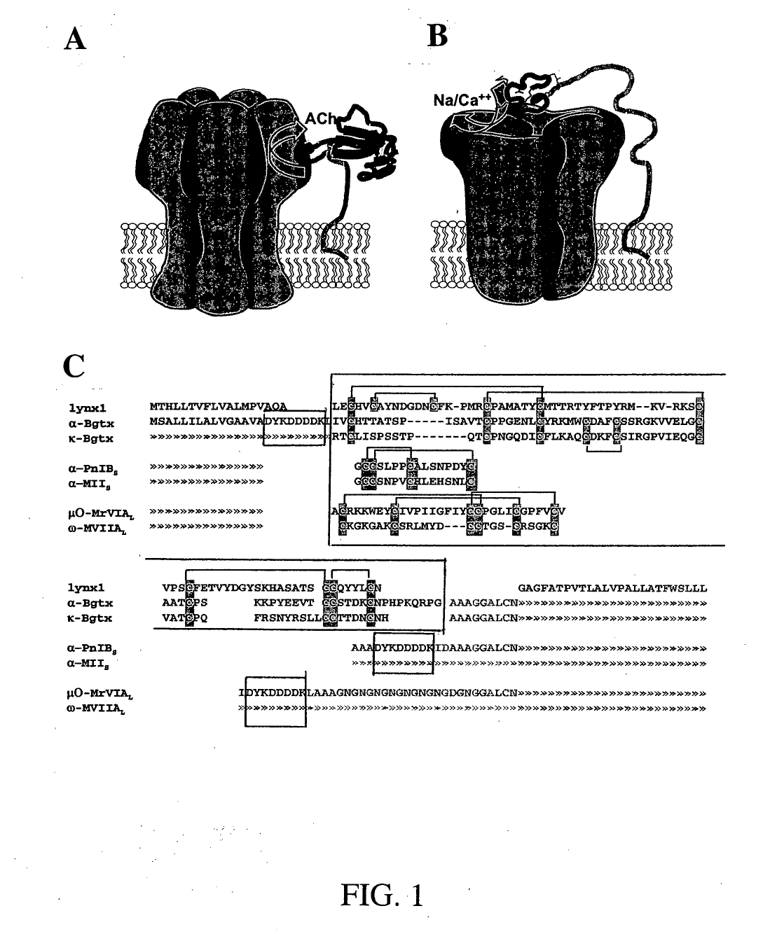

Generation of chimeric tethered toxins: Tethered toxin constructs, containing functional domains from several classes of neurotoxins were expressed on the cell surface using an N-terminal signal peptide, a flexible linker and sequences controlling addition of the lynx1 GPI-anchor (FIG. 1). To test the generality of this approach, tethered toxins specific for several different receptors and ion channels were prepared (TABLE 3). To target nAChRs, we have employed domains from the a and K bungarotoxins, and the α-conotoxins MII and PnIB (Servent et al, 1997; Terlau and Olivera, 2004; Tsetlin and Hucho, 2004). These were chosen because they have distinct specificities for nAChR subtypes, which would therefore allow manipulations of specific classes of these receptors in vivo. A tethered toxin construct carrying the broad spectrum Na⁺ channel inhibitor μ-o-conotoxin MrVIA was also constructed, since this toxin has recently been shown to block rat skeletal muscle and neuronal voltage-gated Na⁺ channels in Xenopus oocytes, and in hippocampal pyramidal neurons (Terlau and Olivera, 2004; Terlau et al, 1996). The activity of this toxin, therefore, could be used to block Na⁺ currents required for action potential generation and propagation. Finally, to target voltage-gated Ca²⁺ channels we have generated a tethered form of the ω-conotoxin MVIIA (Olivera et al, 1994). This toxin can differentiate between N-type, with respect to P/Q-type, L-type and T-type Ca²⁺ channels (Olivera et al, 1994). Specific blockades of Ca²⁺ currents can be used to examine a variety of crucial cellular functions, including mechanisms of pre-synaptic neurotransmitter release, signal transduction, and hormone secretion (Olivera et al, 1994; Tsien, 1983).

TABLE 3
TOXINS USED IN THIS STUDY

| | Receptor/Ion | | | |
|---|---|---|---|---|
| Toxins | Channel | Affinity | | Reference |
| α-bungarotoxin | α1β1γδ | 0.4 | nM | (Lukas et al., 1981) |
| | nAChR | | | |
| | α7nAChR | 0.95 | nM | (Servent et al., 1997) |
| | α4β2 | 100 | μM | (Grutter et al., 2003) |
| | nAChR | | | |
| κ-bungartotoxin | α1β1γδ | 1 | μM | (Loring et al., 1986) |
| | nAChR | | | |
| | α7nAChR | 1-12 | nM | (Servent et al., 1997) |
| | α4β2 | >100 | nM | (Grant et al., 1998) |
| | nAChR | | | |
| α-conotoxin PnlB | α7nAChR | 61.3 | nM | (Luo et al., 1999) |
| α-conotoxin MII | α3β2 | 0.5 | nM | (Cartier et al., 1996) |
| | nAChR | | | |
| μ-o-conotoxin MrVIA | Naᵥ1.2 | 200 | nM | (Terlau et al., 1996) |
| ω-conotoxin MVIIA | Cav2.2 | 1 | nM | (Sato et al., 2000) |

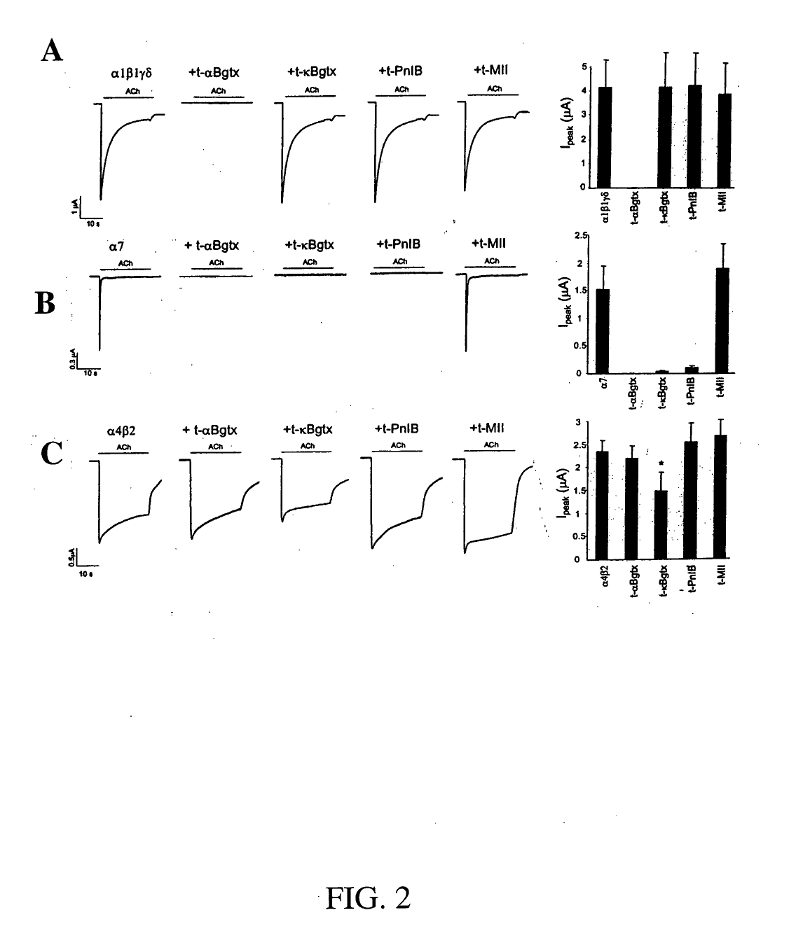

Specific inactivation of nAChR subtypes with tethered bungarotoxins and conotoxins: Each of the tethered toxins was co-injected into Xenopus oocytes with its appropriate target(s) or controls, and assayed by two-electrode voltage clamp recordings. Given the strong structural homology of the bungarotoxins and lynx1 (Miwa et al, 1999), our previous demonstration that lynx1 modulates nAChRs when GPI anchored to the cell surface (Ibanez-Tallon, 2002), and the mode of binding of bungarotoxins to their target receptors (FIG. 1A), it seemed likely that these molecules could retain their activity and specificity when tethered to the cell surface. Recordings of nicotinic receptor activities in response to these tethered toxins confirmed this expectation (FIG. 2).

Thus, co-expression of tethered α-bungarotoxin (t-αBgtx) with muscle α1β1γδ or neuronal α7 nAChRs on the surface of oocytes completely blocks current flow in response to acetylcholine, but does not block α4β2 nAChR function. In contrast, tethered α-bungarotoxin (t-αBgtx) does not block α1βγδ nAChR function when co-expressed in the oocyte system. Rather, it specifically blocks the neuronal nAChR composed of α7subunits, and partially blocks α4β2 receptors, consistent with the relative affinities described in previous studies using soluble toxins (TABLE 3). The fact that the tethered bungarotoxins retained their efficacy and specificity for specific nAChRs prompted us to test whether a second structurally distinct class of toxins known to act on nAChRs could also retain their specificity when tethered to the cell surface (FIG. 2). Thus, tethered conotoxins PnIB (t-PnIB) and MII (t-MII) were tested for their ability to inhibit nAChR currents in oocytes. As shown in FIG. 2, t-PnIB retains its specificity for α7nAChRs, resulting in a block of these currents without affecting either the muscle or neuronal α4β2nAChR activities. As expected (Luo et al, 1999), t-MII does not inhibit the activity of these receptors.

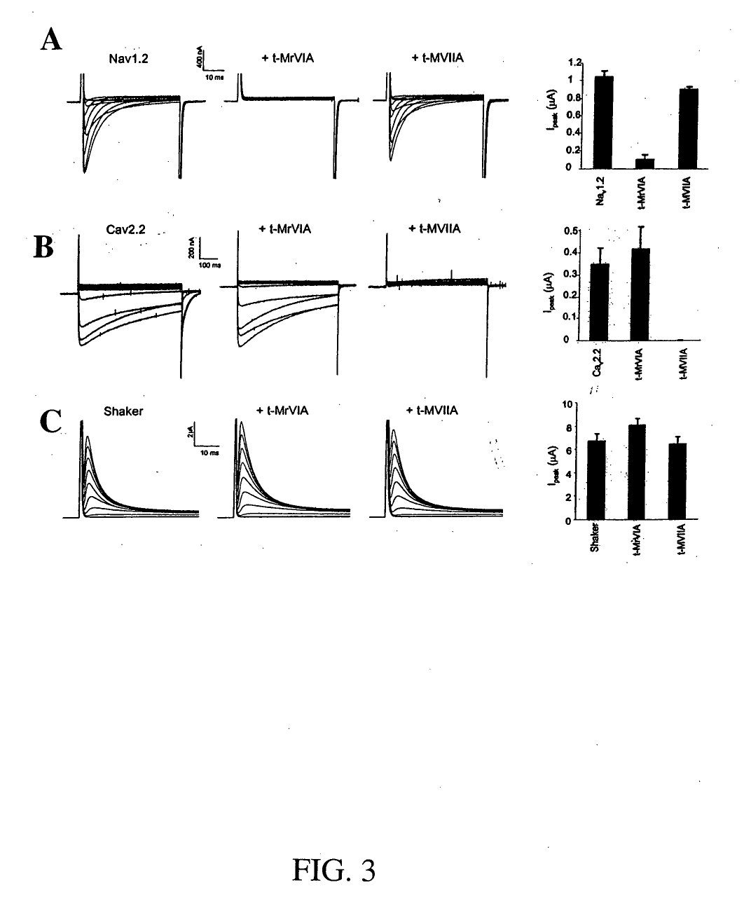

Inactivation of voltage-gated sodium and calcium channels with tethered conotoxins: The ability to use tethered conotoxins to manipulate their cognate targets molecules presented the possibility for performing an astounding array of genetic experiments to manipulate the properties of cells in vivo. Thus, there are approximately 500 species of cone snails, each expressing a unique set of 50-200 peptide toxins, yielding a collection of nearly 50,000 conotoxins, of which less than 0.2% have been characterized (McIntosh et al, 1999). However, a significant proportion of these molecules (e.g. those that block voltage gated ion channels), have been shown to block activity by binding in the vestibule of the ion channel to directly block ion flux (Terlau and Olivera, 2004, Olivera et al, 1994). Given the small size of the conotoxin functional domains, and the increased distance and rotational flexibility required for a tethered toxin to bind properly within the vestibule of the channel (FIG. 1B), it seemed probable that extension of this approach to this class of toxins might be problematic. To investigate this possibility, we next tested tethered toxins directed toward voltage gated Na⁺, and Ca²⁺ channels. In both cases, we have been able to construct tethered toxins that retain both their activity and specificity against their target channels. Thus, the tethered μ-o-conotoxin MrVIA (t-MrVIA) blocked >90% of the current flux through Naᵥ 1.2 channels in oocytes, without affecting either N-type Ca²⁺ channel or shaker K⁺ channel function (FIG. 3). In contrast, tethered 6)-conotoxin MVIIA (t-MVIIA) ω-expression completely blocked Caᵥ2.2 (N-type) Ca²⁺ channels, without effecting either the Naᵥ1.2 Na⁺ or shaker K⁺ channels. Taken together, our data indicate that toxins from several different classes can retain their specificity and activity when expressed as GPI-anchored molecules, effectively inhibiting currents that are fundamental to the physiologic activities of neurons and other cell types. Tethered toxin constructs using toxin molecules that normally contain non-canonical amino acid residues were less successful using canomical amino acid replacements. For example, unsatisfactory results were obtained with several toxins in which trans-4-hydroxyproline is present in the venom, which was substituted by proline in our tethered toxin constructs. Thus, tethered toxin constructs for GID, PIIIA, GVIA and RIIIK all exhibited reduced or no activity when tested in oocytes (data not shown). This may be due to the requirement for hydroxyproline in these toxins, as has been shown in the case of conotoxin GIIIA (Wakamatsu 1992).

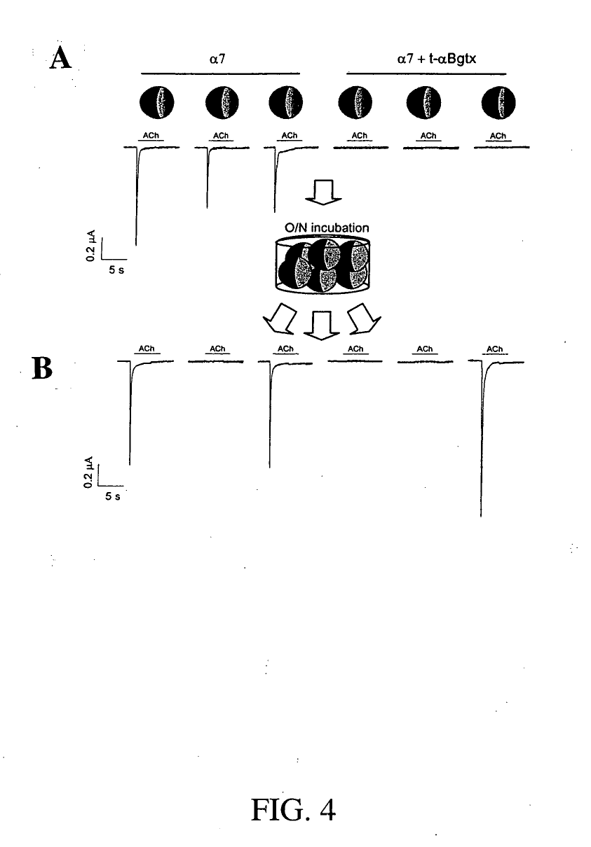

Tethered toxins function cell autonomously: Maximal utility of the tethered toxin approach for in vivo use requires that the toxins act in a cell autonomous manner. To demonstrate that the tethered toxins are not released from the cell surface to affect nearby cells, oocytes co-expressing the tethered α-bungarotoxin construct and neuronal α7 nAChRs were incubated overnight in the presence of oocytes expressing α-7 nAChRs alone (FIG. 4). Each oocyte was then separated from its neighbors, and recordings of nAChR activity in response to acetylcholine measured. As shown in FIG. 4, the receptors on oocytes co-expressing the tethered toxins had no activity in response to ligand, whereas those co-incubated with oocytes expressing only the neuronal α7 nAChR exhibited normal responses to acetylcholine. These data demonstrate that alpha neurotoxins can retain their properties when expressed on the surface of cells via a GPI-anchor, and that they do not affect neighboring cells through release from the cell surface.

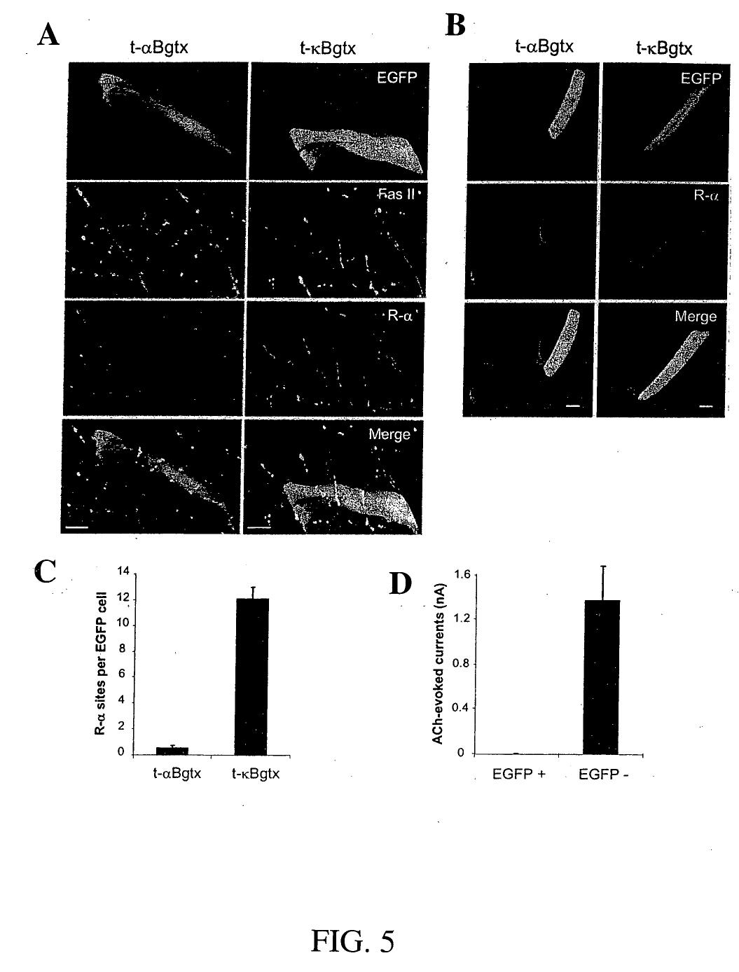

In vivo efficacy of tethered toxins: Zebrafish embryos were chosen to test the efficacy of the tethered toxin approach in vivo because of the ability to visualize single muscle fibers in whole fish and analyze their electrical activity using whole cell recordings. In vivo block of muscle nAChRs by t-αBgtx represents a formidable challenge because of the large number and high density of receptors at the neuromuscular junction. Zebrafish embryos were injected with dual promoter constructs encoding either t-αBgtx or t-κBgtx driven by the muscle specific α-actin promoter and cytoplasmic EGFP driven by the CMV promoter. As shown in FIG. 5A, muscle fibers expressing EGFP were readily identified in fish injected with both tethered toxin constructs. Synaptic sites on EGFP fluorescent muscle cells and their immediate neighbors were identified by labeling with conjugated-fasciculin (FasII) that labels acetylcholinesterase at postsynaptic sites. The positions and morphology of synapses revealed by FasII binding was unaffected in the t-αBgtx and t-κBgtx expressing cells, indicating that the expression of tethered bungarotoxins did not interfere overtly with synapse development. However, muscle cells expressing t-αBgtx had greatly reduced to non-existent levels of soluble rhod-Bgtx labeling, demonstrating that the muscle nicotinic receptors in the t-αBgtx expressing cells were occupied. t-κcpgtx expressing cells retained normal levels of rhod-Bgtx labeling (FIGS. 5A,C). To provide further evidence that the block in rhod-Bgtx labeling was due to occupation of the muscle nicotinic receptors by t-αBgtx, twitch once zebrafish mutants that expressed high levels of the receptor over the entire muscle surface were employed. As shown in FIG. 5B, rhod-Bgtx binding is observed over the surface of control EGFP negative muscle cells, and of EGFP positive cells expressing t-αBgtx, whereas no labeling is detected in EGFP positive cells expressing t-αBgtx. Finally, nAChR function was directly tested in cells expressing t-αBgtx by electrophysiological responses to fast application of 10 μM ACh in acutely dissociated muscle cells from these fish. Peak current in non-fluorescent muscle cells revealed robust responses to ACh (mean current 1.4 nA, n=10), whereas no response was recorded in fluorescent muscle cells co-expressing EGFP and t-αBgtx (mean current 0 nA, n=10) (FIG. 5D). Taken together, these data prove that the tethered bungarotoxins retain their specificity in vivo, that they act cell autonomously, and that t-αBgtx provides an effective block of its cognate receptor in vivo, even under conditions of extremely high receptor expression. These data also indicate that silencing of muscle nicotinic receptor activity in individual muscle cells during zebrafish development has no gross effect on the development or distribution of neuromuscular synapses.

Discussion

We have demonstrated here that peptide neurotoxins from several classes retain their activity and specificity for ligand-gated and voltage-gated ion channels when tethered to the cell membrane via a GPI anchor, and that they act cell autonomously in Xenopus oocytes and zebrafish muscle fibers. The ability to tether these naturally occurring peptide neurotoxins to the cell surface in a manner that preserves their activity and specificity, combined with the use of BAC transgenic constructs to target expression to specific CNS cell types (Gong et al, 2003), allows tremendous flexibility in the genetic dissection of specific factors and pathways that influence development and function of the mammalian CNS. For example, expression of tethered bungarotoxins and α-conotoxins allow simple cell specific and genetic manipulation of specific nAChR classes to begin to unravel the complex contributions of this diverse group of receptors to the activities of specific cell types and circuits in vivo. Also selective suppression of neuronal activity can be achieved using t-MrVIA to inhibit neuronal Na⁺ channels required for generation of the action potential, or t MVIIA to block CA⁺ channels required for neurotransmittal release (Terlau and Olivera 2004). Extension of this approach to other toxins such as those that block other ion channels, serotonin and NMDA receptors offers additional important experimental opportunities.

There are many advantages of the tethered toxin approach over other methods for manipulation of cellular physiology. First, this approach harnesses the impressive functional diversity of the peptide neurotoxins, enabling simple manipulations of ion channels and receptors that mediate important physiologic processes within cells. For example, peptide toxins can differentially block heteromeric channels sharing one or more subunits; conversely, they can block several members of a given ion channel or receptor family. This enables manipulations of currents that would be extremely difficult (or impossible) to achieve using traditional genetic approaches. Second, peptide toxins can act either to block receptors (as in the cases we have chosen for these studies) or to inhibit channel inactivation resulting in hyperexcitation of the target cells, (McIntosh et al, 1999, Terlau and Olivera, 2004), offering the possibility of both loss and gain of function studies in vivo. Third, novel toxin activities can be produced using chimeric toxins derived from fusion of known peptide toxins (Sato et al, 2000). Given the small size of most peptide toxins, we believe that the creation of novel specificities by mutagenesis will extend the use of this approach to receptors and ion channels for which natural toxins have not been identified. Finally, the small size of most tethered toxins and their simple incorporation into transgenic and viral constructs will allow their use in a wide variety of species for which gene targeting is not yet possible (Gong and Rong, 2003). For example, tethered toxins could be employed to examine the influence of receptor function or neuronal activity on behavior in transgenic rats, which are advantageous for certain studies of CNS function.

Two extensions of the present studies are of immediate interest. First, although reversible expression of the tethered toxins can be achieved using established methods (Mansuy and Bujard, 2000), development of strategies for the rapid regulation of these activities for use in short term experiments remains an important goal. Second, the application of this strategy to other peptides, including hormones and neuropeptides, offers interesting opportunities for analysis of the roles of these ligands in specific cell types. Thus, we anticipate that tethered toxins and other peptides will become official instruments for the genetic dissection of CNS cells and circuits.

Experimental Procedures

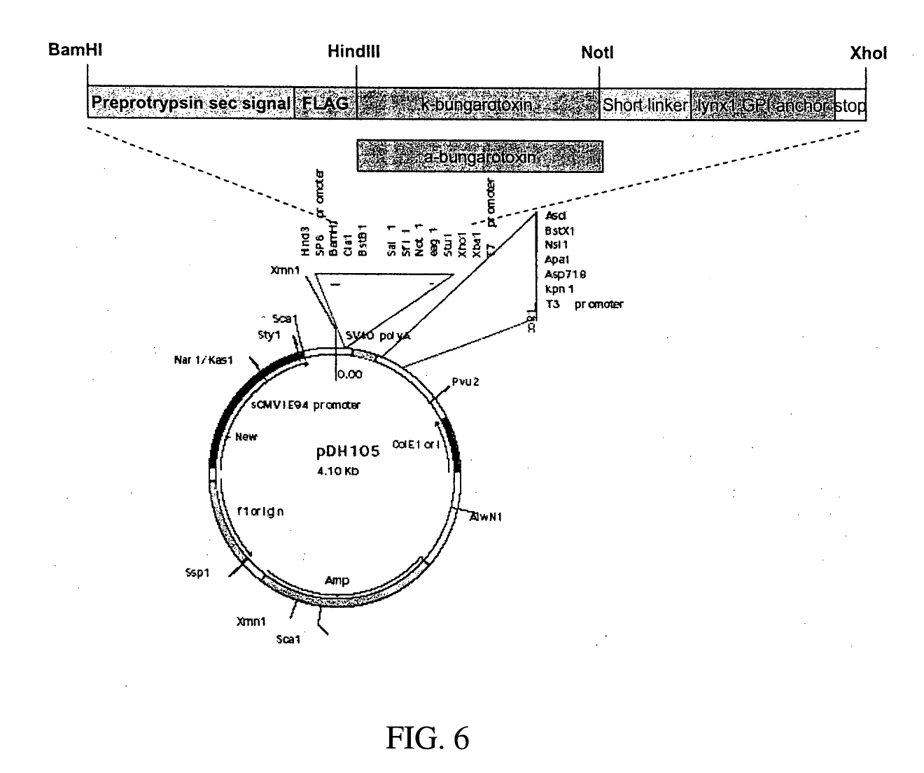

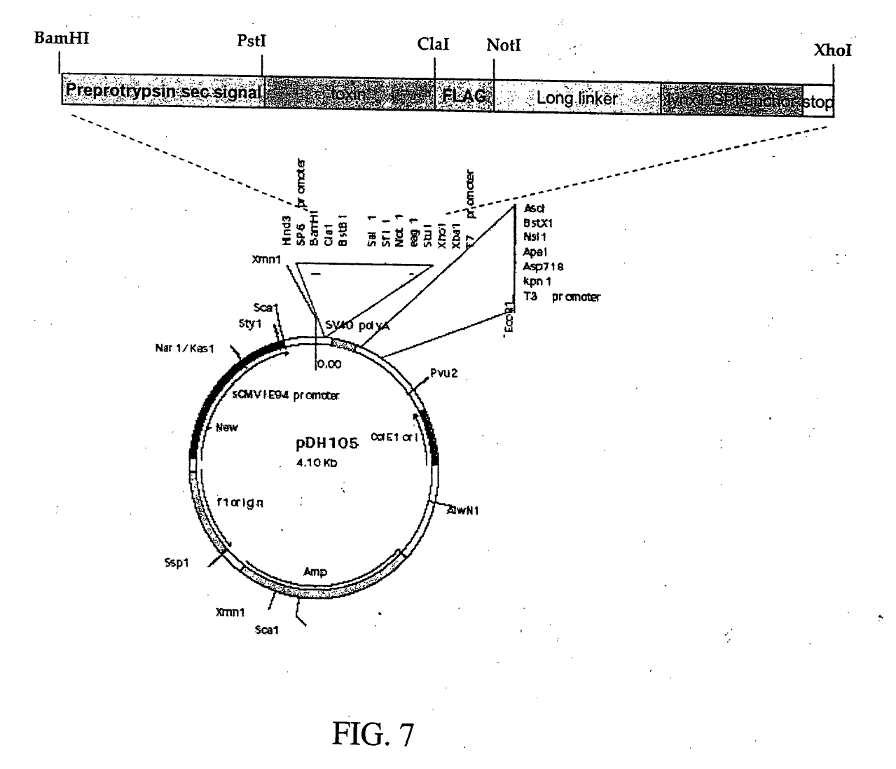

Generation of tethered toxin constructs: Construction of molecular chimeras between lynx1 and the snake bungarotoxins was done by replacing the sequence encoding lynx1 by the cDNAs of α- or κ-bungarotoxin in frame between the secretion signal and lynx1 hydrophobic sequence for GPI attachment. A flag epitope sequence was introduced downstream of the secretion signal and a short 9 amino acid linker was inserted between the toxin and the sequence for GPI attachment. The constructs for tethered conotoxins were prepared as above, except that the flag epitope was inserted downstream of the toxin and that, for toxins against voltage-gated channels, a flexible linker of (asn-gly)ₙ joining the mature toxin molecule to the GPI anchoring sequence was inserted between the toxin and the hydrophobic sequence for GPI attachment. Vectors utilized for these constructs are depicted in FIGS. 6 and 7. The following double stranded oligonucleotides were used to generate the corresponding conotoxins:

PnIB:
5′GGATGTTGCAGTTTACCCCCTTGTGCACTAAGTAACCCGGACTATT
GT3′
MII:
5′GGATGTTGCAGTAATCCAGTATGTCACCTAGAGCATAGCAACCTTT
GC3′
MrVIA:
5′GCATGCCGGAAGAAGTGGGAGTACTGCATAGTGCCGATAATAGGTTCA
TATACTGCTGTCCAGGACTTATATGCGGTCCATTCGTATGCGTC3′
MVIIA:
5′TGCAAAGGCAAGGGCGCGAAGTGCTCCCGCCTCATGTATGACTGTTGC
ACCGGATCGTGTAGGTCCGGTAAGTGC3′

Injections and whole cell electrophysiology recordings: Preparation of Xenopus oocytes, cRNA transcripts, and two-electrode voltage clamp ACh-recordings were done essentially as described (Ibanez-Tallon et al, 2002). cRNA injection mixes containing either receptor/channel subunit cRNAs or the same amount of receptor/channel and t-toxin (t-t) were prepared at the following ratios: for nAChRs [1α1:0.5β1:0.5γ:0.5δ:2t-t], [1α7:3.5t-t], [0.5 α4:0.5β2:3.3 t-t], for sodium Nav1.2 [1α:4t-t] calcium Cav2.2 N-type [1β3:3.2α1β:1α2δ:1t-t] for shaker [1sh: 4t-t]. Recordings of voltage-gated channels were performed as described (Lin et al, 1997; Nadal et al, 2001).

Zebrafish experiments: Tethered bungarotoxins were transiently expressed in zebrafish embryos as described (Ono et al, 2002). The effectiveness of tethered aBgtx to bind and block AChRs in muscle was assessed by in vivo labeling with fluorescence conjugated aBgtx and whole cell recordings (Ono et al, 2001; Ono et al, 2002). Synaptic sites in intact fish were marked by treatment with 0.1 μM Alexa 647 conjugated Fasciculin 2 (Alomone Labs, Israel) for 45 minutes which binds to acetylcholinesterase (Peng et al., 1999). Alternatively, muscle was dissociated incubating skinned fish in 10 mg/ml collagenase (Gibco) for 2 to 3 hours prior to gentle trituration. In some experiments mutant twitch once fish that express large amounts of diffusely distributed acetylcholine receptors were used (Ono et al, 2002). In vivo imaging utilized an inverted Zeiss LSM 510 Meta microscope and a C-Apochromat 40× objective. Simultaneous measurements of EGFP (excitation 488/emission LP505), Alexa-647 (excitation 633/emission LP650) and rhodamine (excitation 543/emission BP560-590) fluorescence were provided by the Meta multi-track mode of acquisition.

### REFERENCES

1. Adams, M. E. and Olivera, B. M. (1994). Neurotoxins: overview of an emerging research technology. Trends Neurosci 17, 151-155.
2. Bauer, E. P., Schafe, G. E. and LeDoux, J.E. (2002). NMDA receptors and L-type voltage-gated calcium channels contribute to long-term potentiation and different components of fear memory formation in the lateral amygdala. J. Neurosci 22, 5239-49.
3. Cartier, G. E., Yoshikami, D., Gray, W.R., Luo, S., Olivera, B. M., and McIntosh, J. M. (1996). A new alpha-conotoxin, which targets alpha3beta2 nicotinic acetylcholine receptors. J. Biol Chem 271, 7522-7528.
4. Catterall, W. A. (1999). Molecular properties of brain sodium channels: an important target for anticonvulsant drugs. Adv Neurol 79, 441-456.
5. Champtiaux, N. and Changeux, J. P. (2004). Knockout and knockin mice to investigate the role of nicotinic receptors in the central nervous system. Prog Brain Res 145, 235-251.
6. Coetzee W A, Amarillo Y, Chiu J, Chow A, Lau D, McCormack T, Moreno H, Nadal M S, Ozaita A, Pountney D, et al. (1999). Molecular diversity of K+ channels. Ann N Y Acad Sci 868, 233-285.
8. Gong, M. and Rong, Y. S. (2003). Targeting multi-cellular organisms. Curr Opin Genet Dev 13, 215-220.
7. Gong, S, Zheng, C., Doughty, M. L., Losos, K., Didkovsky, N., Schambra, U. B., Nowark, N. J., Joyner, A., Leblanc, G., Hatten, M. E., and Heintz, N. (2003). A gene expression atlas of the central nervous system based on bacterial artificial chromosomes. Nature 425, 917-925.
9. Grant, G. A., Luetje, C. W., Summers, R. and Xu, X. L. (1998). Differential roles for disulfide bonds in the structural integrity and biological activity of kappa-Bungarotoxin, a neuronal nicotinic acetylcholine receptor antagonist. Biochemistry 37, 12166-12171.
10. Grutter, T., Prado de Carvalho, L., Le Novere, N., Corringer, P. J., Edelstein, S., and changeux, J. P. (2003). An H-bond between two residues from different loops of the acetylcholine binding site contributes to the activation mechanism of nicotinic receptors. Embo J 22, 1990-2003.
11. Ibanez-Tallon, I., Miwa, J. M., Wang, H. L., Adams, N. C., Crabtree, G. W., Sine, S. M., and Heintz, N. (2002). Novel modulation of neuronal nicotinic acetylcholine receptors by association with the endogenous prototoxin lynxl. Neuron 33, 893-903.
12. Johns, D. C., Marx, R., Mains, R. E., O'Rourke, B., and Marban, E. (1999). Inducible genetic suppression of neuronal excitability. J Neurosci 19, 1691-1697.
13. Kofuji, P., Ceelen, P., Zahs, K. R., Surbeck, L. W., Lester, H. A., and Newman, E. A. (2000). Genetic inactivation of an inwardly rectifying potassium channel (Kir4.1 subunit) in mice: phenotypic impact in retina. J Neurosci 20, 5733-5740.
14. Lin, Z., Haus, S., Edgerton, J. and Lipscombe, D. (1997). Identification of functionally distinct isoforms of the N-type Ca2+ channel in rat sympathetic ganglia and brain. Neuron 18, 153-166.
15. Loring, R. H., Andrews, D., Lane, W. and Zigmond, R. E. (1986). Amino acid sequence of toxin F, a snake venom toxin that blocks neuronal nicotinic receptors. Brain Res 385, 30-37.
16. Lukas, R. J., Morimoto, H., Hanley, M. R. and Bennett, E. L. (1981). Radiolabeled alpha-bungarotoxin derivatives: kinetic interaction with nicotinic acetylcholine receptors. Biochemistry 20, 7373-7378.
17. Luo, S., Nguyen, T. A., Cartier, G. E., Olivera, B. M., Yoshikami, D., and McIntosh, J. M. (1999). Single-residue alteration in alpha-conotoxin PnIA switches its nAChR subtype selectivity. Biochemistry 38, 14542-14548.
18. Manns, J. R., Hopkins, R. O., Reed, J. M., Kitchener, E. G. and Squire, L. R. (2003). Recognition memory and the human hippocampus. Neuron 37, 171-180.
19. Mansuy, I. M., and Bujard, H. (2000). Tetracycline-regulated gene expression in the brain. Curr Opin Neurobiol 10, 593-596.
20. McIntosh, J. M., Santos, A. D., and Olivera, B. M. (1999). Conus peptides targeted to specific nicotinic acetylcholine receptor subtypes. Annu Rev Biochem 68, 59-88.
21. Miwa, J. M., Ibanez-Tallon, I., Crabtree, G. W., Sanchez, R., Sali, A., Role, L. W., and Heintz, N. (1999). lynx 1, an endogenous toxin-like modulator of nicotinic acetylcholine receptors in the mammalian CNS. Neuron 23, 105-114.
22. Nadal, M. S., Amarillo, Y., Vega-Saenz de Miera, E. and Rudy, B. (2001). Evidence for the presence of a novel Kv4-mediated A-type K(+) channel-modifying factor. J Physiol 537, 801-809.
23. Nadeau, H., McKinney, S., Anderson, D. J., and Lester, H. A. (2000). ROMK1 (Kir1.1) causes apoptosis and chronic silencing of hippocampal neurons. J Neurophysiol 84, 1062-1075.
24. Nakazawa, K., McHugh, T. J., Wilson, M. A. and Tonegawa, S. (2004). NMDA receptors, place cells and hippocampal spatial memory. Nat Rev Neurosci 5, 361-372.
25. Nitabach, M. N., Blau, J., and Holmes, T. C. (2002). Electrical silencing of Drosophila pacemaker neurons stops the free-running circadian clock. Cell 109, 485-495.
26. Olivera, B. M., Miljanich, G. P., Ramachandran, J. and Adams, M. E. (1994). Calcium channel diversity and neurotransmitter release: the omega-conotoxins and omega-agatoxins. Annu Rev Biochem 63, 823-867.
27. Ono, F., Higashijima, S., Shcherbatko, A., Fetcho, J. R., and Brehm, P. (2001). Paralytic Zebrafish lacking acetylcholine receptors fail to localize rapsyn clusters to the synapse. J Neurosci 21, 5439-5448.
28. Ono, F., Shcherbatko, A., Higashijima, S., Mandel, G., and Brehm, P. (2002). The Zebrafish motility mutant twitch once reveals new roles for rapsyn in synaptic function. J Neurosci 22, 6491-6498.
29. Paradis, S., Sweeney, S. T., and Davis, G. W. (2001). Homeostatic control of presynaptic release is triggered by postsynaptic membrane depolarization. Neuron 30, 737-749.
30. Peng, H. B., Xie, H., Rossi, S. G., and Rotundo, R. L. (1999). Acetylcholinesterase clustering at the neuromuscular junction involves perlecan and dystroglycan. J Cell Biol 145, 911-921.
31. Saint-Amant L, Drapeau P. (2001). Synchronization of an embryonic network of identified spinal interneurons solely by electrical coupling. Neuron 3, 1035-1046.
32. Sato, K., Raymond, c., Martin-Moutot, N., Sasaki, T., Ohtake, A., Minimai, K., Van Renterghem, C., Takahashi, M., and Seagar, M. J. (2000). Binding of six chimeric analogs of omega-conotoxin MVIIA and MVIIC to N- and P/Q type calcium channels. Biochem Biophys Res Commun 269, 254-256.
33. Servent, D., Winckler-Dietrich, V., Hu, H. Y., Kessler, P., Drevet, P., Bertrand, D., and Menez, A., (1997). Only snake curaremimetic toxins with a fifth disulfide bond have high affinity for the neuronal alpha7 nicotinic receptor. J Biol Chem 272, 24279-24286.
34. Shatz, C. J. and Stryker, M. P. (1978). Ocular dominance in layer IV of the cat's visual cortex and the effects of monocular deprivation. J Physiol 281, 267-283.
35. Shatz, C. J. and Stryker, M. P. (1988). Prenatal tetrodotoxin infusion blocks segregation of retinogeniculate afferents. Science 242, 87-89.
36. Steinhardt R A, Bi G, Alderton J M. (1994). Cell membrane resealing by a vesicular mechanism similar to neurotransmitter release. Science 263, 390-393.
37. Terlau, H. and Olivera, B. M. (2004). Conus venoms: a rich source of novel ion channel-targeted peptides. Physiol Rev 84, 41-68.
38. Terlau, H., Stocker, M., Shon, K. J., McIntosh, J. M. and Olivera, B. M. (1996). MicroO-conotoxin MrVIA inhibits mammalian sodium channels, but not through site I. J Neurophysiol 76, 1423-1429.
39. Tsetlin, V. I. and Hucho, F. (2004). Snake and snail toxins acting on nicotinic acetylcholine receptors: fundamental aspects and medical applications. FEBS Lett 557, 9-13.
40. Tsien, R. W. (1983). Calcium channels in excitable cell membranes. Annu Rev Physiol 45, 341-358.
41. Wakamatsu, K., Kohda, D., Hatanaka, H., Lancelin, J. M., Ishida, Y., Oya, M., Nakamura, H., Inagaki, F., and Sato, K. (1992). Structure-activity relationships of mu-conotoxin GIIIA: structure determination of active and inactive sodium channel blocker peptides by NMR and simulated annealing calculations. Biochemistry 31, 12577-12584.

### EXAMPLE 2

We have generated plasmids useful to clone any toxin using particular restriction sites. These generic tethered toxin cassettes or expression constructs are based on a pDH105 vector backbone and are depicted in FIGS. 6 and 7. In FIG. 6, a plasmid is shown for tethering a toxin using Hind III or Not 1 restriction sites, whereby a cassette containing, for example α, or κ-bungarotoxin, followed by a linker and GPI anchoring sequence is inserted after the preprotoxin secretion signal and Flag epitope. This toxin insert is cloned into the oocyte vector CS2 using Ban HI and XhoI sites. The vector depicted in FIG. 7 uses Pst 1 and Cla I sites for the toxin, inserting the toxin between the preprotoxin secretion signal and the FLAG epitope and adding a longer linker between the FLAG epitope and the GPI anchor sequence.

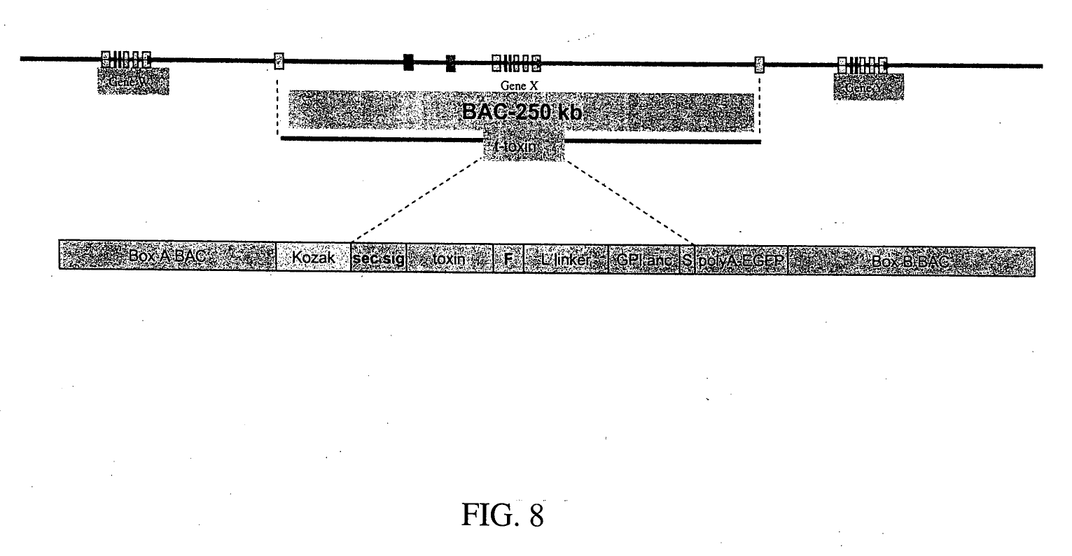

A generic constrict for generating bacterial artificial chromosome (BAC) transgenics expressing tethered toxins is depicted in FIG. 8. The tethered toxin construct is cloned between a KOZAK sequence and polyA sequence (in this instance that of EGFP), which are needed for proper expression in vivo. Box A and Box B sequences, corresponding to the DNA of the gene used to target cell-specific expression, are cloned flanking the tethered toxin.

### EXAMPLE 3

### Retrovirus Encoded Thethered Bungarotoxin

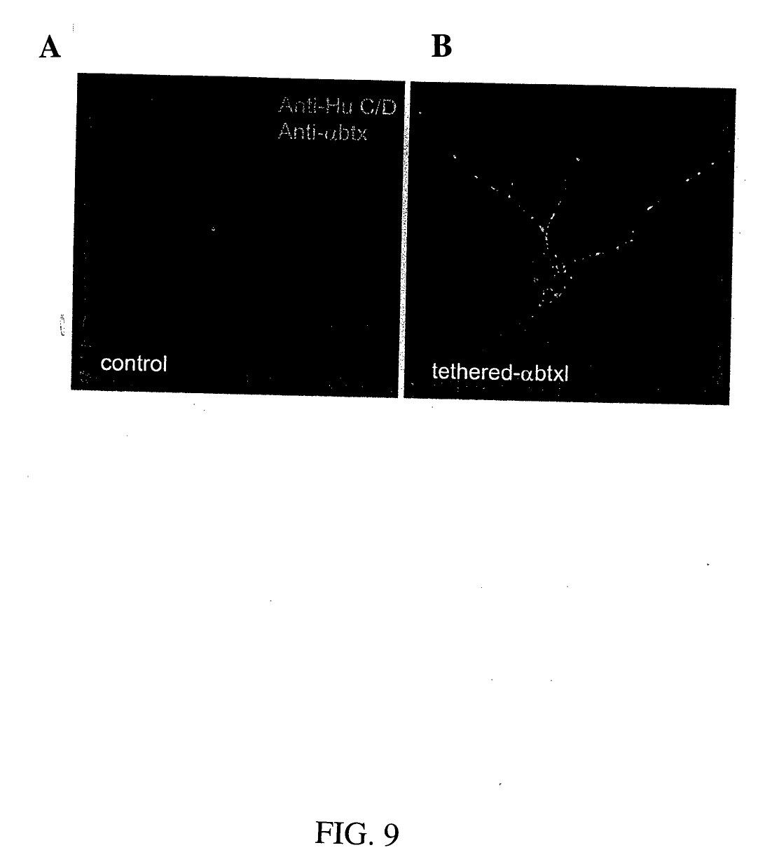

Infection of chick ciliary ganglion neurons with retrovirus encoded tethered α-bungarotoxin demonstrates that tethered α-btx is expressed by antibody staining. The GPI-anchored α-bungarotoxin was cloned into ClaI restriction sites of the shuttle vector (pS1ax12). That construct was then used for subcloning into the chicken retroviral vector RCASBP(A) (“Manipulating Gen Expression with Replication-Competent Retroviruses”, Chapter 10 in Methods in Cell Biology (1996) 51:185-218). Chick embryos were injected with concentrated retrovirus RCASBP(A)-tethered-αbtx at St 8-9. Ciliary ganglia were isolated at St 34 (E8), dissociated and plated into cell culture on glass coverslips. After 24 hours, cells were incubated live on ice with a rabbit antibody anti-αbtx (gift of Dr. Joshua Sanes, Washington University), washed, fixed, then incubated in anti-Hu C/D (Molecular Probes) to identify neuron, and the appropriate secondary antibodies, Cy3 goat anti-rat (Jackson Laboratories) and Alexa488 goat anti-mouse (Molecular Probes). Cells were photographed using confocal microscopy (FIG. 9). In cultures from control ganglia infected with RCASBP vector alone (FIG. 9, left panel) cell bodies are labeled red for Hu but are unlabeled with anti-αbtx. In contrast, tethered-αbtx-expressing neurons bind anti-αbtx over their cell bodies and processes (FIG. 9, right panel), indicating that the tethered-αbtx is expressed on the neuronal cell surface.

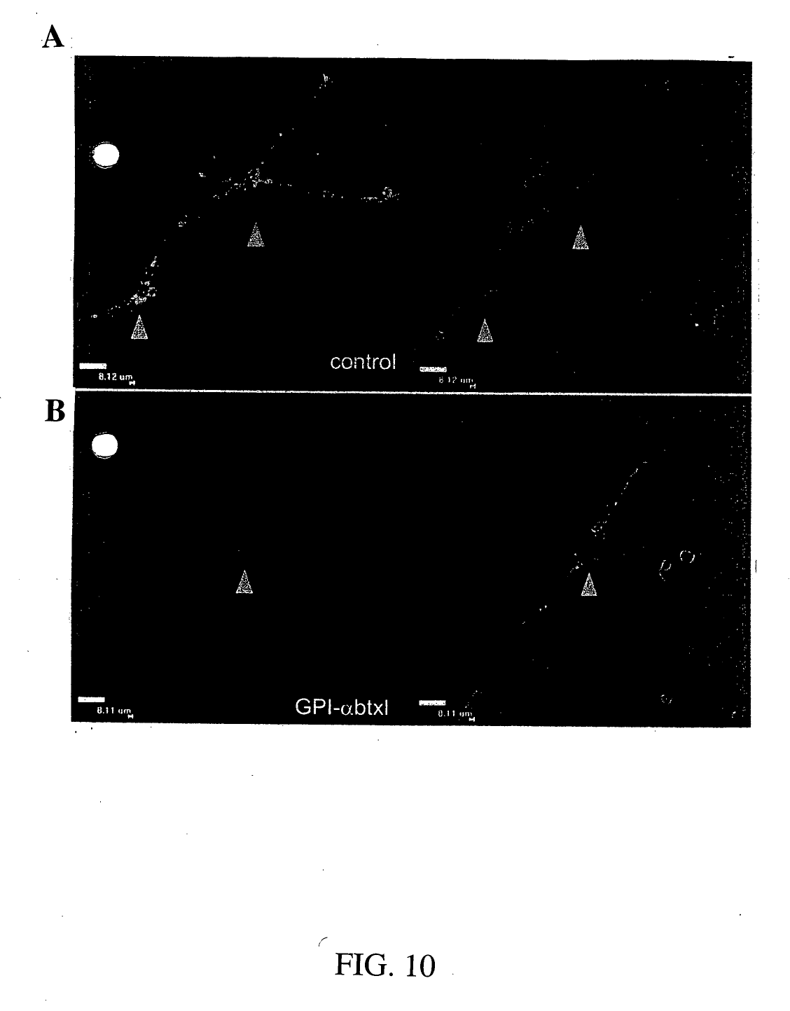

The expression of tethered αbtx was confirmed in retrovirus infected ciliary ganglia by binding competition of Alexa conjugated αbtx. Ciliary ganglia were isolated from St 34 chick embryos injected with retrovirus RCASBP(A)-tethered-αbtx is discussed above and plated into cell culture on glass overslips. After an overnight incubation, cells were incubated in 1 μg/ml Alexa 488-αbtx (Molecular Probes, Cat. No. B13422), washed, then photographed using epifluorescence microscopy (FIG. 10). Control neurons stain brightly with the αbtx, but the tethered-αbtx-expressing neurons do not because the virally expressed construct occupies all the α7 nAChRs.

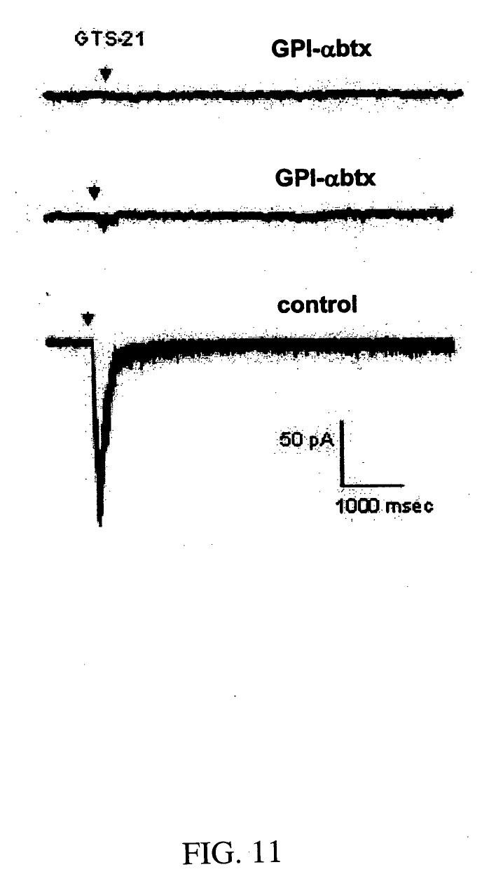

In the chick embryos expressing retrovirus encoded tethered-αbtx, it was next demonstrated that expression of tethered-αbtx in neurons blocks nicotinic receptor function. Again ciliary ganglia from retrovirus RCASBP(A) tethered-αbtx infected chick embryos were isolated, dissociated and plated into cell culture on 15 mm glass coverslips. Three hours after plating, the response of neurons to the α7-nAChR specific agonist GTS-21 (Nai et al (2003) Molec Pharm 63:311) was assessed by whole cell patch clamp (FIG. 11). FIG. 11 A and B represent the responses of two different tethered-αbtx infected neurons. Overall, recordings were obtained from 11 tethered-αbtx infected neurons and 13 control neurons in two different injection experiments. Agonist was applied using a puffer pipette containing 300 μM GTS-21. While response was observed in control neurons, no significant response was observed in tethered-αbtx infected neurons.

### EXAMPLE 4

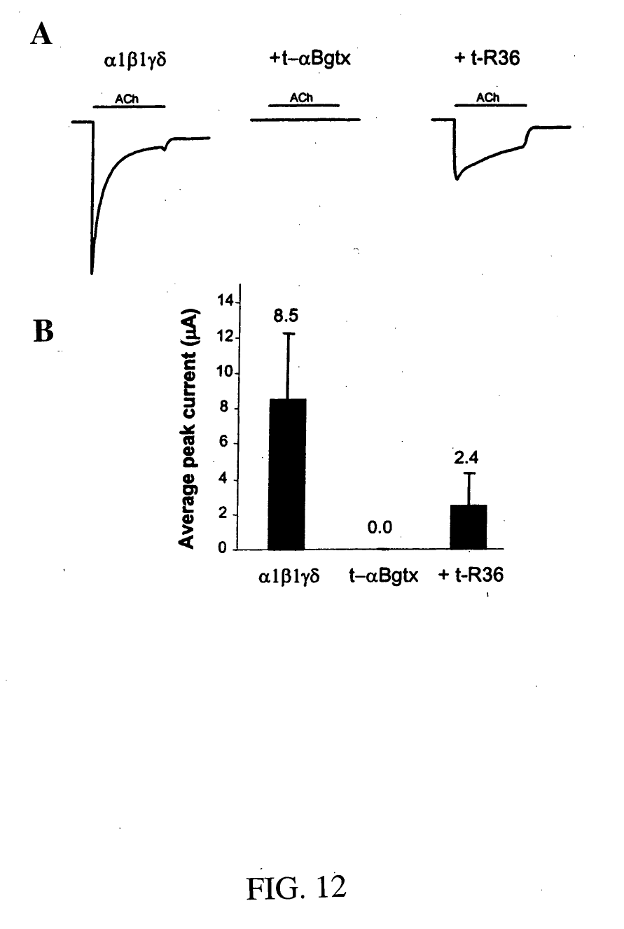

A mutant of α-bungarotoxin was generated and expressed as a tethered α-bungarotoxin. A point mutation deletion of arg 361 (t-R36) was created and expressed in tethered form. This arginine residue is located at the central loop of α-bungarotoxin. Xenopus oocytes were coinjected with the muscle nicotinic receptor α1β1γδ alone or together with tethered α-bungarotoxin (+t-αpgtx) or with tethered mutant α-bungarotoxin (+t-R36). The electrophysiology recordings (FIG. 12A) show that while tethered α-bungarotoxin completely blocks the receptor response to ACh, the t-R36 mutant only partially blocks the currents. In addition, the kinetics of the ACh response currents are changed in the presence of the t-R36 mutant. FIG. 12A depicts a fast desensitization response on expression of nicotinic receptor alone, compared to a slower desensitization response in the presence of t-R36. The average peak currents for the α1β1γδ receptor alone (8.46 +/−3.7 μA), receptor plus t-αβgtx (0 +/−0 μA), and receptor plus t-26 (2.4 +/−1.8 μA) are graphed in FIG. 12B. These results provide evidence of the application of the tethered toxin methodology to the construction and expression of new or altered synthetic toxins with new or altered properties.

This invention may be embodied in other forms or carried out in other ways without departing from the spirit or essential characteristics thereof. The present disclosure is therefore to be considered as in all aspects illustrate and not restrictive, the scope of the invention being indicated by the appended claims, and all changes which come within the meaning and range of equivalency are intended to be embraced therein.

Various references are cited throughout this Specification, each of which is incorporated herein by reference in its entirety.

What is claimed is:

1. A method for tethering toxins or peptides to a surface comprising attaching to said toxin or peptide a heterologous sequence from a membrane protein or cell surface protein which is naturally attached to or traverses the cell membrane and expressing said toxin or peptide in tethered form.

2. The method of claim 1 wherein the heterologous sequence is a membrane attachment sequence.

3. The method of claim 2 wherein the membrane attachment sequence is selected from the group of a transmembrane domain, a hydrophobic domain, a PH domain, a GPI attachment sequence, a myristoylation sequence (Cys-A-A-X) (SEQ ID NO:1), and a palmitoylation sequence.

4. The method of claim 1 wherein the toxin or peptide is not naturally tethered or attached to the cell surface or through a cell membrane.

5. The method of claim 1 wherein the toxin or peptide is selected from the group of a neuropeptide, an immune modulator, a cytokine, a hormone, a conotoxin, a bungarotoxin, a spider neuroactive toxin, a snake neuroactive toxin, a scorpion neuroactive toxin, a snail neuroactive toxin, a bacterial neuroactive toxin, a bee neuroactive toxin and a fish neuroactive toxin.

6. The method of claim 1 wherein the toxin or peptide is a mutant or altered peptide sequence or non-peptide toxin.

7. The method of claim 1 wherein the surface is a cell surface or membrane.

8. The method of claim 1 wherein the peptide or toxin is expressed in tethered form in and on the cell expressing its receptor or ion channel.

9. The method of claim 1 wherein the peptide or toxin is expressed in tethered form in and on a distinct cell, not expressing its receptor or ion channel.

10. A tethering cassette for expression of or preparation of a tethered peptide, which peptide is not naturally tethered or attached to the cell surface or through a cell membrane, comprising a promoter, a peptide to be tethered, a linker sequence and a membrane attachment sequence.

11. The cassette of claim 10 further comprising a means for induction or modulation of the expression or activity of the tethered peptide.

12. The cassette of claim 10 further comprising a secretion signal located in the cassette prior to the peptide to be tethered.

13. The cassette of claim 10 wherein the toxin or peptide is selected from the group of a neuropeptide, an immune modulator, a cytokine, a hormone, a conotoxin, a bungarotoxin, a spider neuroactive toxin, a snake neuroactive toxin, a scorpion neuroactive toxin, a snail neuroactive toxin, a bacterial neuroactive toxin, a bee neuroactive toxin and a fish neuroactive toxin.

14. The cassette of claim 10 wherein the membrane attachment sequence is selected from the group of a transmembrane domain, a hydrophobic domain, a PH domain, a GPI attachment sequence, a myristoylation sequence (Cys-A-A-X) (SEQ ID NO:1), and a palmitoylation sequence.

15. A recombinant DNA molecule or cloned gene which encodes a tethered toxin or peptide comprising a nucleic acid sequence encoding a toxin or peptide which is not naturally tethered, membrane bound or membrane associated, a linker sequence and a membrane attachment sequence selected from the group of a transmembrane domain, a hydrophobic domain, a PH domain, a GPI attachment sequence, a myristoylation sequence (Cys-A-A-X) (SEQ ID NO:1), and a palmitoylation sequence.

16. The recombinant DNA molecule of claim 15 wherein the toxin or peptide is selected from the group of a neuropeptide, an immune modulator, a cytokine, a hormone, a conotoxin, a bungarotoxin, a spider neuroactive toxin, a snake neuroactive toxin, a scorpion neuroactive toxin, a snail neuroactive toxin, a bacterial neuroactive toxin, a bee neuroactive toxin and a fish neuroactive toxin.

17. A tethered toxin or peptide, which is not naturally tethered, membrane bound or membrane associated, the toxin or peptide capable of attaching to or tethering to a surface and capable of interacting with, signaling via, or otherwise modulating at least one of its natural cell receptors or ion channels, wherein the toxin or peptide is modified by attachment of a heterologous sequence from a membrane protein or cell surface protein which is naturally attached to or traverses the cell membrane.

18. The tethered toxin or peptide of claim 17 wherein the toxin or peptide is selected from the group of a neuropeptide, an immune modulator, a cytokine, a hormone, a conotoxin, a bungarotoxin, a spider neuroactive toxin, a snake neuroactive toxin, a scorpion neuroactive toxin, a snail neuroactive toxin, a bacterial neuroactive toxin, a bee neuroactive toxin and a fish neuroactive toxin.

19. The tethered toxin or peptide of claim 18 wherein the heterologous sequence is a membrane attachment sequence and is selected from the group of a transmembrane domain, a hydrophobic domain, a PH domain, a GPI attachment sequence, a myristoylation sequence (Cys-A-A-X) (SEQ ID NO:1), and a palmitoylation sequence.

20. A method or assay system for screening of potential drugs effective to modulate the activity of a receptor in mammalian cells whereby a toxin or peptide capable of interacting with the receptor is expressed in tethered form in the mammalian cells or on a surface or cell interacting with the mammalian cells, wherein the toxin or peptide is tethered by attachment of a heterologous sequence from a membrane protein or cell surface protein which is naturally attached to or traverses the cell membrane and wherein the ability of the drug to modulate the receptor is assessed by measuring its ability to interrupt or potentiate the tethered peptides.
